(19) United States
(12) Patent Application Publication
(10) Pub. No.: US 2015/0173830 A1
(43) Pub. Date: Jun. 25, 2015

(54) TREATMENT STRUCTURE AND METHODS OF USE

(71) Applicants: Eric JOHNSON, Woodside, CA (US); Jeremy STIGALL, Carlsbad, CA (US)

(72) Inventors: Eric JOHNSON, Woodside, CA (US); Jeremy STIGALL, Carlsbad, CA (US)

(21) Appl. No.: 14/581,638

(22) Filed: Dec. 23, 2014

Related U.S. Application Data
(60) Provisional application No. 61/920,320, filed on Dec. 23, 2013.

(51) Int. Cl. A61B 18/14; A61B 5/06; A61F 7/12; A61B 8/08; A61F 7/00; A61B 18/02

(52) U.S. Cl. CPC A61B 18/1492; A61F 7/007; A61B 18/0218; A61F 7/12; A61B 18/1402; A61B 8/0841; A61B 5/061; A61F 2007/126; A61B 2018/0212; A61B 2018/00583

(57) ABSTRACT

Catheter based ablation devices, systems, and methods for achieving intraluminal and/or transluminal neuromodulation, stimulation or ablation by intraluminal access are disclosed herein. One aspect of the present application, for example, is directed to devices, systems, and methods that incorporate a spiral support frame treatment device on an elongate shaft or as a temporary or permanently implanted therapy device. The elongated shaft is sized and configured to deliver a spiral treatment device to a treatment position via an intravascular path. Intraluminal and transluminal neuromodulation, stimulation or ablation or other therapeutic outcomes may be achieved via energy delivered via the spiral element. One therapy includes modulation of neural fibers that contribute to or alter vascular structures that feed or perfuse the neural fibers.

### TREATMENT STRUCTURE AND METHODS OF USE

This application claims priority to U.S. Provisional Patent Application Ser. No. 61/920,320 titled “Treatment Structure and Methods of Use,” filed on Dec. 23, 2013, which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

The technologies disclosed in the present application generally relate to catheter apparatuses, systems, and methods for intraluminal or transluminal neuromodulation, neurostimulation or ablation. More particularly, the technologies disclosed herein relate to therapy elements borne by a spiral frame device utilized in systems and methods for providing intraluminal and transluminal therapy as well as specific uses for intravascular or transvascular therapy. In one aspect, for example, devices and methods of using those devices are applied to neural structures accessed via the renal arteries.

### BACKGROUND

There have been a number of systems proposed for treatments of intravascular and transvascular therapy modes, particularly for accessing the renal arteries. Many of these systems, however, are challenged by difficulty in proper positioning of a therapy element or delivery of appropriate energy patterns at levels for the intended therapeutic purpose.

The sympathetic nervous system (SNS) is a primarily involuntary bodily control system typically associated with stress responses. Fibers of the SNS innervate tissue in almost every organ system of the human body and can affect characteristics such as pupil diameter, gut motility, and urinary output. Such regulation can have adaptive utility in maintaining homeostasis or in preparing the body for rapid response to environmental factors. Chronic activation of the SNS, however, is a common maladaptive response that can drive the progression of many disease states. Excessive activation of the renal SNS in particular has been identified as a likely contributor to the complex pathophysiology of hypertension, states of volume overload (such as heart failure), and progressive renal disease. For example, radiotracer dilution has demonstrated increased renal norepinephrine (NE) spillover rates in patients with essential hypertension.

Cardio-renal sympathetic nerve hyperactivity can be particularly pronounced in patients with heart failure. For example, an exaggerated NE overflow from the heart and kidneys to plasma is often found in these patients. Heightened SNS activation commonly characterizes both chronic and end stage renal disease. In patients with end stage renal disease, NE plasma levels above the median have been demonstrated to be predictive for cardiovascular diseases and several causes of death. This is also true for patients suffering from diabetic or contrast nephropathy. Evidence suggests that sensory afferent signals originating from diseased kidneys are major contributors to initiating and sustaining elevated central sympathetic outflow.

Sympathetic nerves innervating the kidneys terminate in the blood vessels, the juxtaglomerular apparatus, and the renal tubules. Stimulation of the renal sympathetic has been proposed in a number of systems. However, challenges remain for proper positioning of therapy elements and efficacious treatments. It would be desirable to provide methods and apparatus for achieving improved devices, system and methodologies for intraluminal and transluminal placement of therapy devices and related therapies. Accordingly, a need remains for alternative treatment strategies.

SUMMARY OF THE DISCLOSURE

In general, in one embodiment, a spiral support frame therapy delivery device, includes a first spiral support member; a second spiral support member positioned adjacent to the first spiral support member to form a first loop, a second loop and a crossover between the first loop and the second loop; at least one therapy element borne by the first spiral support member or the second spiral support member wherein an outer surface of the at least one therapy element used for contacting a tissue to be treated is positioned in relation to an outer surface of the first spiral support member or the second spiral support member; an energy source in communication with the at least one therapy element adapted and configured to deliver a therapy via the at least one therapy element.

This and other embodiments can include one or more of the following features. In one aspect, the first spiral support frame can be coupled to the second support frame at the crossover. In another aspect, the outer surface of the at least one therapy element used for contacting a tissue to be treated can be positioned at about the same level in relation to an outer surface of the first spiral support member or the second spiral support member. In a further aspect, the outer surface of the at least one therapy element used for contacting a tissue to be treated can be positioned below the level of an outer surface of the first spiral support member or the second spiral support member. In an alternative aspect, the outer surface of the at least one therapy element used for contacting a tissue to be treated can be positioned above the level of an outer surface of the first spiral support member or the second spiral support member. In yet another aspect, the first spiral support member and the second spiral support member can be disposed within a lumen of a multiple lumen sheath and the at least one therapy element can be in communication with the energy source via a suitable connection placed with or along a lumen a multiple lumen sheath. In still another aspect, the support members and the suitable connection can be the same. In one aspect, the support members can be in a lumen separated from the lumen containing the at least one therapy element and the suitable connection to the energy source. In another aspect, the first spiral support member and the second spiral support member can be each disposed within a first lumen of multiple lumen sheath, the at least one therapy element can be in communication with the energy source via a suitable connection placed within or along a second lumen a multiple lumen sheath and a syringe or pump or reservoir containing a therapy agent can be in communication with a third lumen of a multiple lumen sheath and to one or more perfusion apertures formed in a sidewall of the third lumen. In a further aspect, the first lumen can be positioned within about the center of the multiple lumen sheath. In an alternative aspect, the first lumen can be positioned within about the center of the multiple lumen sheath and the second lumen and the third lumen can be positioned to one side of the first lumen.

In yet another aspect, the first lumen, the second lumen and the third lumen can be positioned so as to be about evenly spaced about the sheath interior. In still another aspect, the first lumen, the second lumen and the third lumen can be positioned so as to be unevenly spaced about the sheath interior. In one aspect, the first spiral support member and the second spiral support member can be each disposed within a first lumen of multiple lumen sheath, the at least one therapy element can be in communication with the energy source via a suitable connection placed within or along a second lumen a multiple lumen sheath and a third lumen of a multiple lumen sheath including one or more apertures formed in a sidewall of the third lumen further including a syringe or pump or reservoir containing a therapy agent is in communication with the third lumen and the one or more apertures or one or more fixation elements contained within the third lumen and deployable through the one or more apertures formed in the sidewall of the third lumen. In another aspect, the first spiral support member and the second spiral support member can be each disposed within a first lumen of multiple lumen sheath, the at least one therapy element can be in communication with the energy source via a suitable connection placed within or along a second lumen a multiple lumen sheath and a third lumen of a multiple lumen sheath including one or more apertures formed in a sidewall of the third lumen further including a syringe or pump or reservoir containing a therapy agent is in communication with the third lumen and the one or more apertures; a fourth lumen of a multiple lumen sheath including one or more apertures formed in a sidewall of the fourth lumen and one or more fixation elements contained within the fourth lumen and deployable through the one or more apertures formed in the sidewall of the fourth lumen.

In general, in one embodiment, an intraluminal treatment apparatus, including an elongated shaft having a proximal portion in communication with an energy source and a distal portion coupled to a spiral support frame; at least one treatment element borne by the spiral support frame and connected via the elongated shaft to the energy source, wherein the support frame is configured for intravascular delivery to a transluminal therapy site in a patient; and wherein the treatment element is configured for positioning against an interior wall of the lumen to modulate, stimulate or ablate a neural tissue at the transluminal treatment site in the patient.

This and other embodiments can include one or more of the following features. In one aspect, the at least one treatment device can be positioned on one side of a crossover of the spiral support frame. In another aspect, the at least one treatment device can further include at least one treatment device on a first spiral support member of the spiral support frame and at least one treatment device on a second spiral support member of the spiral support frame. In a further aspect, the at least one treatment device can further include at least one treatment device on a first spiral support member including a plurality of individually controlled treatment devices on a first spiral support member of the spiral support frame and at least one treatment device on the second spiral support member including a plurality of individually controlled treatment devices on a second spiral support member of the spiral support frame.

In one embodiment, there is provided a treatment apparatus, comprising an elongated shaft having a proximal portion, a distal portion having a spiral support frame; at least one treatment element borne by the support frame, wherein the support frame is configured for intravascular delivery to a renal artery of a human patient; and wherein the treatment element is configured for positioning against a wall of the renal artery to modulate a renal nerve at a treatment site within the renal artery.

In one aspect, there is provided a spiral support frame therapy delivery device, comprising a first spiral support member; a second spiral support member positioned adjacent to the first spiral support member to form a first loop, a second loop and a moving crossover between the first loop and the second loop; at least one therapy element borne by the first spiral support member or the second spiral support member; a therapy delivery device in communications with the at least one therapy element adapted and configured to deliver a therapy via the at least one therapy element.

In another embodiment, there is provided a method for spiral support frame based renal neuromodulation, comprising positioning a spiral support frame based having a therapeutic element within a renal artery of a human patient; and reducing neural traffic to and/or from a kidney of the patient via the therapeutic element, wherein reducing the neural traffic to and/or from the kidney therapeutically treats a diagnosed condition or disease associated with cardio-renal function of the patient.

In still another aspect, there are also devices and systems adapted and configured for positioning a spiral support frame within a patient using one or more of percutaneously, intravascularly, endovascularly or interventionally positioning the spiral support frame within a lumen, a cavity, an interstitial space, an organ, within an implant site, within a bone, with a joint or on, in or above any of the above described anatomical sites.

In still other embodiments, there is described a method for spiral support frame-based renal denervation comprising positioning a spiral support frame having a therapeutic element within a renal artery of a human patient; and ablating nerves that innervate a kidney of the patient via the therapeutic element, wherein ablating the nerves results in a therapeutically beneficial reduction in blood pressure in the patient. The method may be advantageously used in the delivery of one or modes of therapy into one or more treatment locations within a patient corresponding to a treatment scheme for the treatment of: a patient diagnosed with hypertension, a patient diagnosed with heart failure, a patient diagnosed with acute myocardial infarction, a patient diagnosed with kidney disease, a patient diagnosed with chronic renal failure, and/or a patient diagnosed with a chronically activated sympathetic nervous system.

In still other embodiments, there is a method for spiral support frame based renal denervation comprising positioning a spiral support frame having a therapeutic element within the vasculature of a human patient; and ablating nerves that innervate a portion of the vasculature of the patient via the therapeutic element, wherein ablating the nerves results in a therapeutically beneficial reduction in a targeted central sympathetic overactivity in the patient. In other advantageous aspects, the therapeutically beneficial reduction in central sympathetic overactivity comprises a reduction in clinical symptoms of hypertension in the patient, a reduction in clinical symptoms of heart failure in the patient, a reduction in clinical symptoms of chronic kidney disease in the patient, a reduction in clinical symptoms of end stage renal disease in the patient, and/or a reduction in clinical symptoms of acute myocardial infarction in the patient.

In still another embodiment, there is provided an implantable treatment apparatus having an elongated shaft having a proximal portion, a distal portion having a spiral support frame; at least one treatment element borne by the support frame, wherein the support frame is configured for delivery to and implantation within a human patient adjacent to a treatment location; and wherein the treatment element is configured for positioning against a portion of the anatomy of the patient in a position suited to the therapeutic range or therapy modality of the at least one treatment element borne by the support frame. In one aspect, the treatment location is accessible and the support frame is configured for delivery via an artery or a vein. In still another aspect, the treatment location is accessible and the support frame is configured for delivery via a minimally invasive, laparoscopic or arthroscopic or single port access delivery system. Still further, the treatment location is accessible and the support frame is configured for delivery via a lumen or natural orifice of the patient.

In still another embodiment, there is a spiral support frame therapy delivery device having a first spiral support member; a second spiral support member positioned adjacent to the first spiral support member to form a first loop, a second loop and a moving crossover between the first loop and the second loop; at least one therapy element borne by the first spiral support member or the second spiral support member; a therapy delivery device in communications with the at least one therapy element adapted and configured to deliver a therapy via the at least one therapy element. In one aspect, the treatment location is accessible and the support frame is configured for delivery via an artery or a vein. In still another aspect, the treatment location is accessible and the support frame is configured for delivery via a minimally invasive, laparoscopic or arthroscopic or single port access delivery system.

In still other aspects, there is also a method for spiral support frame based stimulation, neuromodulation, or ablation, comprising positioning a spiral support frame based having a therapeutic element within the vasculature of a human patient; and reducing neural traffic to and/or from or along one or more nerves adjacent the vasculature via the therapeutic element, wherein reducing the neural traffic to and/or from or along the nerves therapeutically treats a diagnosed condition or disease associated with the one or more nerves of the patient.

In other aspects, any of the above described aspects, alternatives or embodiments may also provide for positioning a spiral support frame so that positioning of an electrode or a therapeutic element within the vasculature places the electrode or a therapeutic element in contact with a wall of the vasculature without distention of the wall, with mild distention of the wall or with substantial distention of the wall. In some aspects, the degree of distention is used to position the electrode or a therapeutic element in proximity to a target structure adjacent to the wall. In other aspects, the degree of distention is used to adjust the apposition of the electrode or a therapeutic element with the wall. In still further aspects, monitoring, increasing or decreasing contact between an electrode or a therapeutic element and the wall of the vasculature or a lumen or an implant location within the body is provided by adjusting the radical forces of the spiral support frame, selecting an oversized support frame or adjusting an delivery device adapted and configured to increase the radial force on the support frame.

In still other embodiments, there is provided techniques for positioning a spiral support frame therapeutic device within a patient using a catheter, using fluoroscopic guidance or using intra vascular ultrasound, an endoscope or other suitable delivery modality or imaging modality. In still further aspects, there is also provided advantageous positioning a single or multiple treatment mode spiral support frame within an vein, an artery, a lumen, a cavity or an implant site within the patient through one or a combination of percutaneously, intravascularly, endovascularly or interventionally positioning the spiral support frame within the patient. In still other aspects and alteratives there is also a method for spiral support frame-based therapy accomplished by positioning a spiral support frame having at least one therapeutic element within a human patient to a treatment site; operating at least one therapeutic element for a treatment delivery in at least one therapy mode; and controlling the operation of the at least one therapeutic element according to a predetermined therapeutically beneficial outcome for a diagnosed condition of the patient. Still further, there is a method for spiral support frame-based therapy including positioning within a human patient to a treatment site a spiral support frame therapy device having at least one therapeutic element for operation in a first therapy mode and at least one therapy element for operation in a second therapy mode; operating the spiral support frame therapy device for the delivery in at least one therapy mode; and controlling the operation of the spiral support frame therapy device according to a predetermined therapeutically beneficial outcome for a diagnosed condition of the patient.

In still other aspects of any of the above, the spiral support frame therapy device remains implanted within the patient and is operated via an implanted or an external control scheme. In additional or alternative aspects, the embodiments or aspects described above may also include an internally implanted or an external controller adapted and configured for wireless connectivity to the device for control or monitoring of the device or an internally implanted or and externally positioned controller adapted and configured for a wired connectivity to the device for control or monitoring of the device.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 is a conceptual illustration of the sympathetic nervous system (SNS) and how the brain communicates with the body via the SNS.

FIG. 2 is an enlarged anatomic view of nerves innervating a left kidney to form the renal plexus surrounding the left renal artery.

FIGS. 3A and 3B provide, respectively, anatomic and conceptual views of a human body, depicting neural efferent and afferent communication between the brain and kidneys.

FIGS. 4A and 4B are, respectively, anatomic views of the arterial and venous vasculatures of a human.

FIG. 5 illustrates a therapy delivery system configured for providing treatments according to embodiments described herein.

FIG. 6A illustrates a therapy device embodiment described herein positioned to provide therapy to a renal artery.

FIGS. 6B, 6C and 6D illustrate an enlarged section view of FIG. 6A having a treatment device in position to delivery therapy in a renal artery with varying levels of apposition to the vessel wall.

FIG. 7 is a flow chart for an exemplary method of providing therapy.

FIGS. 8A and 8B illustrate, respectively, the positioning and the first stage of deployment for a therapy device within a vessel.

FIG. 9 illustrates an enlarged view of a cross over section of therapy device and a pair of treatment elements in contact with the lumen wall.

FIG. 10A illustrates a treatment device having a therapy element along the length of one support frame sized for a treatment position within a lumen.

FIG. 10B illustrates a treatment device having multiple therapy elements spaced along the length of one support frame sized for a treatment position within a lumen.

FIG. 10C illustrates a treatment device having multiple therapy elements spaced along only a portion of the length of one support frame sized for a treatment position within a lumen.

FIGS. 11A and 11B illustrate a support frame having a plurality of treatment elements that fully encircle a portion of the support frame, with the support frame in a deployed and a stowed configuration, respectively.

FIG. 11C illustrates a support frame according to FIG. 11A but having a plurality of treatment elements that only partially encircle a portion of the support frame.

FIG. 11D illustrates a support frame as in FIGS. 11A and 11C having treatment elements only on one portion of the support frame.

FIGS. 12A-12E illustrate various views of the relationship between one or more therapy elements and a support frame. FIG. 12A illustrates a cross section view of a round support frame element and a round surface mounted therapy element.

FIG. 12B illustrates a cross section view of a rectangular support frame element and an embedded therapy element.

FIG. 12C illustrates a cross section view of a rectangular support frame element and a pair of oval shaped therapy elements partially embedded into the support frame element.

FIG. 12D illustrates a cross section view of a curved support frame element and a rectangular shaped therapy element embedded into the support frame element.

FIG. 12E illustrates a perspective view of a round support frame element and a ring shaped therapy element about the support frame element.

FIG. 12F illustrates a perspective view of a portion of a support frame having a linear array of regularly sized and spaced therapy elements along a portion of a support frame.

FIG. 12G illustrates a perspective view of a portion of a support frame having an irregular dispersion pattern of similarly sized therapy elements along a portion of a support frame.

FIG. 12H illustrates a perspective view of a portion of a support frame having a pair of spaced apart penetrating therapy elements.

FIG. 13A illustrates a multiple loop support frame having a therapy element extending from the proximal end to the distal end of the support frame.

FIG. 13B illustrates a multiple loop support frame having a plurality of therapy elements placed only on one of the support frame loops.

FIG. 14 illustrates a perspective view of a multiple lumen support structure having a support frame in one conduit and therapy element access provided by another conduit.

FIG. 15 illustrates a perspective view of a multiple lumen support structure having a support frame completely within one lumen and a series of therapy elements provided within a portion of a segmented lumen.

FIG. 15A illustrate cross section view of the therapy element and segmented lumen of FIG. 15 with a therapy element positioned in a recessed configuration relative to the segmented lumen.

FIG. 15B illustrate cross section view of the therapy element and segmented lumen of FIG. 15 with a therapy element in an in-line configuration relative to the segmented lumen.

FIG. 15C illustrate cross section view of the therapy element and segmented lumen of FIG. 15 with a therapy element positioned in a protruding configuration relative to the segmented lumen.

FIGS. 16A and 16B are perspective and cross section views, respectively, of a multiple conduit multiple therapy mode treatment device configured to have having a support frame conduit, a treatment element conduit and a perfusion conduit.

FIGS. 16C, 16D, 16E and 16F illustrate cross section views of various multiple conduit multiple therapy mode treatment devices.

FIGS. 17A and 17B illustrate perspective and enlarged partial views, respectively, of a multiple conduit multiple therapy mode treatment device configured to have having a support frame conduit, a treatment element conduit, a perfusion conduit and an anchoring element conduit.

FIG. 18A illustrates a multiple conduit, multiple therapy mode treatment device in position to delivery therapy in the left renal artery.

FIG. 18B illustrates a pair of multiple conduit, multiple therapy mode treatment device separately placed in position to delivery therapy in the left and the right renal arteries.

FIG. 18C illustrates a pair of multiple conduit, multiple therapy mode treatment device placed in position to delivery therapy in the left and the right renal arteries using a single delivery catheter.

FIG. 19 is a flow chart for an exemplary method of providing therapy utilizing a multiple therapy mode device.

### DETAILED DESCRIPTION

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof as used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements, these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

The present technology is directed to apparatuses, systems, and methods for achieving electrically—and/or thermally-induced and/or chemically induced renal neuromodulation (i.e., rendering neural fibers that innervate the kidney inert or inactive or otherwise completely or partially reduced in function) by percutaneous transluminal intravascular access. In particular, embodiments of the present technology relate to apparatuses, systems, and methods that incorporate a spiral support frame catheter based treatment device. The catheter treatment device may comprise an elongated shaft sized and configured to deliver at least one therapy element borne by a spiral support frame within a renal artery via an intravascular path (e.g., a femoral artery, an iliac artery and the aorta, a transradial approach, or another suitable intravascular path). In one embodiment, for example, the therapy element delivers energy directly to the renal artery wall in order to induce heating of target renal nerves or of vascular structures that perfuse target renal nerves. The suitable generator may be positioned along or near a proximal region of the elongated shaft external to the patient, while suitable delivery lines extend along or within the elongated shaft, to the distal region of the shaft configured for therapy element placement in the renal artery via the intravascular path.

I. Pertinent Anatomy and Physiology

The following discussion provides various details regarding pertinent patient anatomy and physiology. This section is intended to provide additional context regarding the disclosed technology and the therapeutic benefits associated with renal denervation, and to supplement and expand upon the disclosure herein regarding the relevant anatomy and physiology. For example, as mentioned below, several properties of the renal vasculature may inform the design of treatment devices and associated methods for achieving renal neuromodulation via intravascular access, and impose specific design requirements for such devices. Specific design requirements may include accessing the renal artery, facilitating stable contact between the energy delivery elements of such devices and a luminal surface or wall of the renal artery, and/or effectively modulating the renal nerves with the neuromodulatory apparatus.

A. The Sympathetic Nervous System

The Sympathetic Nervous System (SNS) is a branch of the autonomic nervous system along with the enteric nervous system and parasympathetic nervous system. It is always active at a basal level (called sympathetic tone) and becomes more active during times of stress. Like other parts of the nervous system, the sympathetic nervous system operates through a series of interconnected neurons. Sympathetic neurons are frequently considered part of the peripheral nervous system (PNS), although many lie within the central nervous system (CNS). Sympathetic neurons of the spinal cord (which is part of the CNS) communicate with peripheral sympathetic neurons via a series of sympathetic ganglia. Within the ganglia, spinal cord sympathetic neurons join peripheral sympathetic neurons through synapses. Spinal cord sympathetic neurons are therefore called presynaptic (or preganglionic) neurons, while peripheral sympathetic neurons are called postsynaptic (or postganglionic) neurons.

At synapses within the sympathetic ganglia, preganglionic sympathetic neurons release acetylcholine, a chemical messenger that binds and activates nicotinic acetylcholine receptors on postganglionic neurons. In response to this stimulus, postganglionic neurons principally release noradrenaline (norepinephrine). Prolonged activation may elicit the release of adrenaline from the adrenal medulla.

Once released, norepinephrine and epinephrine bind adrenergic receptors on peripheral tissues. Binding to adrenergic receptors causes a neuronal and hormonal response. The physiologic manifestations include pupil dilation, increased heart rate, occasional vomiting, and increased blood pressure. Increased sweating is also seen due to binding of cholinergic receptors of the sweat glands.

The sympathetic nervous system is responsible for up-and down-regulating many homeostatic mechanisms in living organisms. Fibers from the SNS innervate tissues in almost every organ system, providing at least some regulatory function to things as diverse as pupil diameter, gut motility, and urinary output. This response is also known as sympatho-adrenal response of the body, as the preganglionic sympathetic fibers that end in the adrenal medulla (but also all other sympathetic fibers) secrete acetylcholine, which activates the secretion of adrenaline (epinephrine) and to a lesser extent noradrenaline (norepinephrine). Therefore, this response that acts primarily on the cardiovascular system is mediated directly via impulses transmitted through the sympathetic nervous system and indirectly via catecholamines secreted from the adrenal medulla.

Science typically looks at the SNS as an automatic regulation system, that is, one that operates without the intervention of conscious thought. Some evolutionary theorists suggest that the sympathetic nervous system operated in early organisms to maintain survival as the sympathetic nervous system is responsible for priming the body for action. One example of this priming is in the moments before waking, in which sympathetic outflow spontaneously increases in preparation for action.

1. The Sympathetic Chain

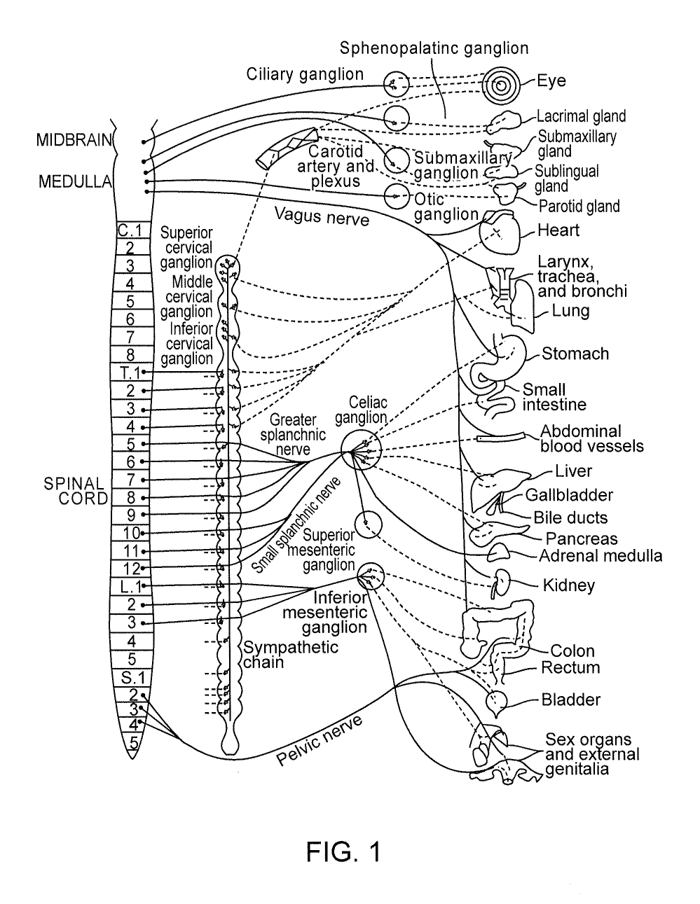

As shown in FIG. 1, the SNS provides a network of nerves that allows the brain to communicate with the body. Sympathetic nerves originate inside the vertebral column, toward the middle of the spinal cord in the intermediolateral cell column (or lateral horn), beginning at the first thoracic segment of the spinal cord and are thought to extend to the second or third lumbar segments. Because its cells begin in the thoracic and lumbar regions of the spinal cord, the SNS is said to have a thoracolumbar outflow. Axons of these nerves leave the spinal cord through the anterior rootlet/root. They pass near the spinal (sensory) ganglion, where they enter the anterior rami of the spinal nerves. However, unlike somatic innervation, they quickly separate out through white rami connectors which connect to either the paravertebral (which lie near the vertebral column) or prevertebral (which lie near the aortic bifurcation) ganglia extending alongside the spinal column.

In order to reach the target organs and glands, the axons should travel long distances in the body, and, to accomplish this, many axons relay their message to a second cell through synaptic transmission. The ends of the axons link across a space, the synapse, to the dendrites of the second cell. The first cell (the presynaptic cell) sends a neurotransmitter across the synaptic cleft where it activates the second cell (the postsynaptic cell). The message is then carried to the final destination.

In the SNS and other components of the peripheral nervous system, these synapses are made at sites called ganglia. The cell that sends its fiber is called a preganglionic cell, while the cell whose fiber leaves the ganglion is called a postganglionic cell. As mentioned previously, the preganglionic cells of the SNS are located between the first thoracic (T1) segment and third lumbar (L3) segments of the spinal cord. Postganglionic cells have their cell bodies in the ganglia and send their axons to target organs or glands.

The ganglia include not just the sympathetic trunks but also the cervical ganglia (superior, middle and inferior), which sends sympathetic nerve fibers to the head and thorax organs, and the celiac and mesenteric ganglia (which send sympathetic fibers to the gut).

2. Innervation of the Kidneys

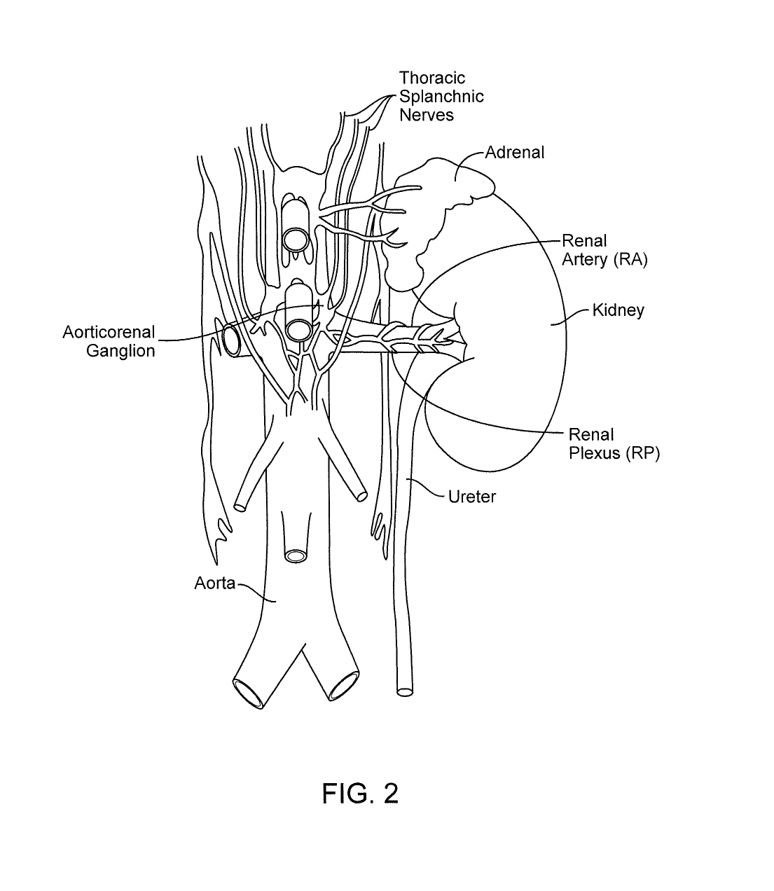

As FIG. 2 shows, the kidney is innervated by the renal plexus RP, which is intimately associated with the renal artery. The renal plexus RP is an autonomic plexus that surrounds the renal artery and is embedded within the adventitia of the renal artery. The renal plexus RP extends along the renal artery until it arrives at the substance of the kidney. Fibers contributing to the renal plexus RP arise from the celiac ganglion, the superior mesenteric ganglion, the aorticorenal ganglion and the aortic plexus. The renal plexus RP, also referred to as the renal nerve, is predominantly comprised of sympathetic components. There is no (or at least very minimal) parasympathetic innervation of the kidney.

Preganglionic neuronal cell bodies are located in the intermediolateral cell column of the spinal cord. Preganglionic axons pass through the paravertebral ganglia (they do not synapse) to become the lesser splanchnic nerve, the least splanchnic nerve, first lumbar splanchnic nerve, second lumbar splanchnic nerve, and travel to the celiac ganglion, the superior mesenteric ganglion, and the aorticorenal ganglion. Postganglionic neuronal cell bodies exit the celiac ganglion, the superior mesenteric ganglion, and the aorticorenal ganglion to the renal plexus RP and are distributed to the renal vasculature.

3. Renal Sympathetic Neural Activity

Messages travel through the SNS in a bidirectional flow. Efferent messages may trigger changes in different parts of the body simultaneously. For example, the sympathetic nervous system may accelerate heart rate; widen bronchial passages; decrease motility (movement) of the large intestine; constrict blood vessels; increase peristalsis in the esophagus; cause pupil dilation, piloerection (goose bumps) and perspiration (sweating); and raise blood pressure. Afferent messages carry signals from various organs and sensory receptors in the body to other organs and, particularly, the brain.

Hypertension, heart failure and chronic kidney disease are a few of many disease states that result from chronic activation of the SNS, especially the renal sympathetic nervous system. Chronic activation of the SNS is a maladaptive response that drives the progression of these disease states. Pharmaceutical management of the renin-angiotensin-aldosterone system (RAAS) has been a longstanding, but somewhat ineffective, approach for reducing over-activity of the SNS.

As mentioned above, the renal sympathetic nervous system has been identified as a major contributor to the complex pathophysiology of hypertension, states of volume overload (such as heart failure), and progressive renal disease, both experimentally and in humans. Studies employing radiotracer dilution methodology to measure overflow of norepinephrine from the kidneys to plasma revealed increased renal norepinephrine (NE) spillover rates in patients with essential hypertension, particularly so in young hypertensive subjects, which in concert with increased NE spillover from the heart, is consistent with the hemodynamic profile typically seen in early hypertension and characterized by an increased heart rate, cardiac output, and renovascular resistance. It is now known that essential hypertension is commonly neurogenic, often accompanied by pronounced sympathetic nervous system overactivity.

Activation of cardiorenal sympathetic nerve activity is even more pronounced in heart failure, as demonstrated by an exaggerated increase of NE overflow from the heart and the kidneys to plasma in this patient group. In line with this notion is the recent demonstration of a strong negative predictive value of renal sympathetic activation on all-cause mortality and heart transplantation in patients with congestive heart failure, which is independent of overall sympathetic activity, glomerular filtration rate, and left ventricular ejection fraction. These findings support the notion that treatment regimens that are designed to reduce renal sympathetic stimulation have the potential to improve survival in patients with heart failure.

Both chronic and end stage renal disease are characterized by heightened sympathetic nervous activation. In patients with end stage renal disease, plasma levels of norepinephrine above the median have been demonstrated to be predictive for both all-cause death and death from cardiovascular disease. This is also true for patients suffering from diabetic or contrast nephropathy. There is compelling evidence suggesting that sensory afferent signals originating from the diseased kidneys are major contributors to initiating and sustaining elevated central sympathetic outflow in this patient group; this facilitates the occurrence of the well known adverse consequences of chronic sympathetic over activity, such as hypertension, left ventricular hypertrophy, ventricular arrhythmias, sudden cardiac death, insulin resistance, diabetes, and metabolic syndrome.

(i) Renal Sympathetic Efferent Activity

Sympathetic nerves to the kidneys terminate in the blood vessels, the juxtaglomerular apparatus and the renal tubules. Stimulation of the renal sympathetic nerves causes increased renin release, increased sodium (Na+) reabsorption, and a reduction of renal blood flow. These components of the neural regulation of renal function are considerably stimulated in disease states characterized by heightened sympathetic tone and clearly contribute to the rise in blood pressure in hypertensive patients. The reduction of renal blood flow and glomerular filtration rate as a result of renal sympathetic efferent stimulation is likely a cornerstone of the loss of renal function in cardio-renal syndrome, which is renal dysfunction as a progressive complication of chronic heart failure, with a clinical course that typically fluctuates with the patient's clinical status and treatment. Pharmacologic strategies to thwart the consequences of renal efferent sympathetic stimulation include centrally acting sympatholytic drugs, beta blockers (intended to reduce renin release), angiotensin converting enzyme inhibitors and receptor blockers (intended to block the action of angiotensin II and aldosterone activation consequent to renin release) and diuretics (intended to counter the renal sympathetic mediated sodium and water retention). However, the current pharmacologic strategies have significant limitations including limited efficacy, compliance issues, side effects and others.

(ii) Renal Sensory Afferent Nerve Activity

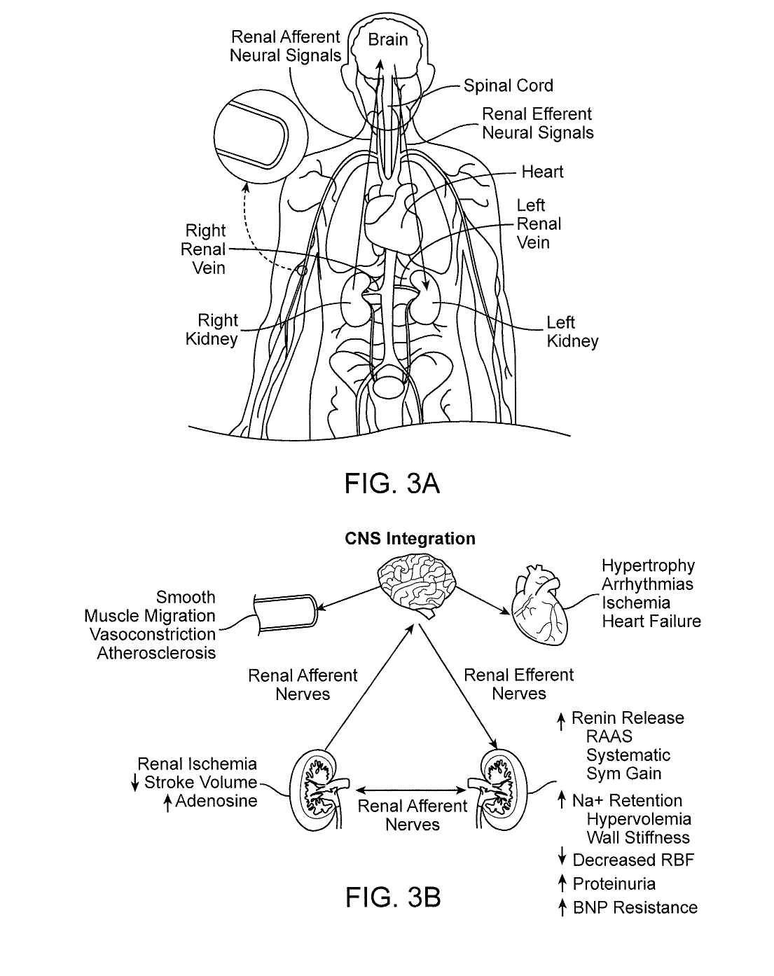

The kidneys communicate with integral structures in the central nervous system via renal sensory afferent nerves. Several forms of “renal injury” may induce activation of sensory afferent signals. For example, renal ischemia, reduction in stroke volume or renal blood flow, or an abundance of adenosine enzyme may trigger activation of afferent neural communication. As shown in FIGS. 3A and 3B, this afferent communication might be from the kidney to the brain or might be from one kidney to the other kidney (via the central nervous system). These afferent signals are centrally integrated and may result in increased sympathetic outflow. This sympathetic drive is directed towards the kidneys, thereby activating the RAAS and inducing increased renin secretion, sodium retention, volume retention and vasoconstriction. Central sympathetic over activity also impacts other organs and bodily structures innervated by sympathetic nerves such as the heart and the peripheral vasculature, resulting in the described adverse effects of sympathetic activation, several aspects of which also contribute to the rise in blood pressure.

The physiology therefore suggests that (i) modulation of tissue with efferent sympathetic nerves will reduce inappropriate renin release, salt retention, and reduction of renal blood flow, and that (ii) modulation of tissue with afferent sensory nerves will reduce the systemic contribution to hypertension and other disease states associated with increased central sympathetic tone through its direct effect on the posterior hypothalamus as well as the contralateral kidney. In addition to the central hypotensive effects of afferent renal denervation, a desirable reduction of central sympathetic outflow to various other sympathetically innervated organs such as the heart and the vasculature is anticipated.

B. Additional Clinical Benefits of Renal Denervation

As provided above, renal denervation is likely to be valuable in the treatment of several clinical conditions characterized by increased overall and particularly renal sympathetic activity such as hypertension, metabolic syndrome, insulin resistance, diabetes, left ventricular hypertrophy, chronic end stage renal disease, inappropriate fluid retention in heart failure, cardio-renal syndrome, and sudden death. Since the reduction of afferent neural signals contributes to the systemic reduction of sympathetic tone/drive, renal denervation might also be useful in treating other conditions associated with systemic sympathetic hyperactivity. Accordingly, renal denervation may also benefit other organs and bodily structures innervated by sympathetic nerves, including those identified in FIG. 1. For example, as previously discussed, a reduction in central sympathetic drive may reduce the insulin resistance that afflicts people with metabolic syndrome and Type II diabetics. Additionally, patients with osteoporosis are also sympathetically activated and might also benefit from the down regulation of sympathetic drive that accompanies renal denervation.

C. Achieving Intravascular Access to the Renal Artery

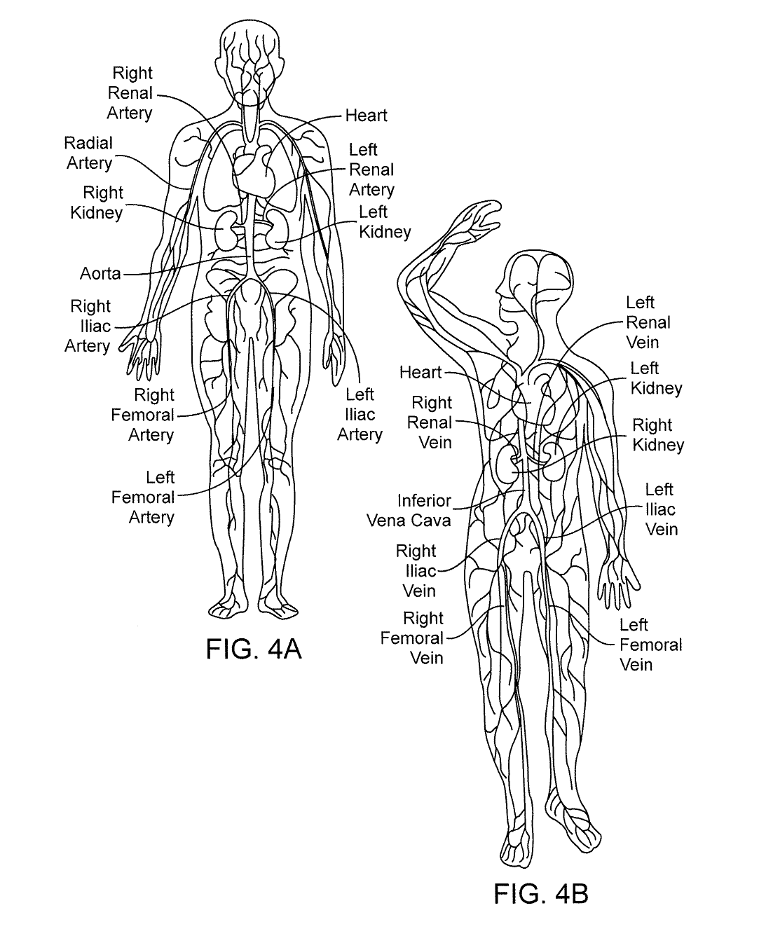

In accordance with the present technology, neuromodulation of a left and/or right renal plexus RP, which is intimately associated with a left and/or right renal artery, may be achieved through intravascular access. As FIG. 4A shows, blood moved by contractions of the heart is conveyed from the left ventricle of the heart by the aorta. The aorta descends through the thorax and branches into the left and right renal arteries. Below the renal arteries, the aorta bifurcates at the left and right iliac arteries. The left and right iliac arteries descend, respectively, through the left and right legs and join the left and right femoral arteries.

As FIG. 4B shows, the blood collects in veins and returns to the heart, through the femoral veins into the iliac veins and into the inferior vena cava. The inferior vena cava branches into the left and right renal veins. Above the renal veins, the inferior vena cava ascends to convey blood into the right atrium of the heart. From the right atrium, the blood is pumped through the right ventricle into the lungs, where it is oxygenated. From the lungs, the oxygenated blood is conveyed into the left atrium. From the left atrium, the oxygenated blood is conveyed by the left ventricle back to the aorta.

As will be described in greater detail later, the femoral artery may be accessed and cannulated at the base of the femoral triangle just inferior to the midpoint of the inguinal ligament. A catheter may be inserted percutaneously into the femoral artery through this access site, passed through the iliac artery and aorta, and placed into either the left or right renal artery. This comprises an intravascular path that offers minimally invasive access to a respective renal artery and/or other renal blood vessels.

The wrist, upper arm, and shoulder region provide other locations for introduction of catheters into the arterial system. For example, catheterization of either the radial, brachial, or axillary artery may be utilized in select cases. Catheters introduced via these access points may be passed through the subclavian artery on the left side (or via the subclavian and brachiocephalic arteries on the right side), through the aortic arch, down the descending aorta and into the renal arteries using standard angiographic technique.

D. Properties and Characteristics of the Renal Vasculature

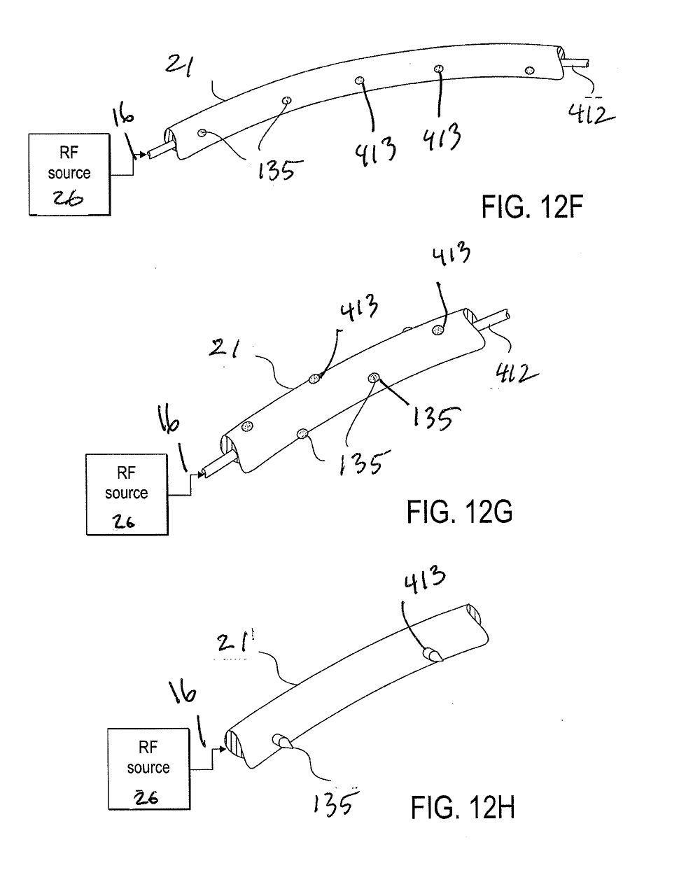

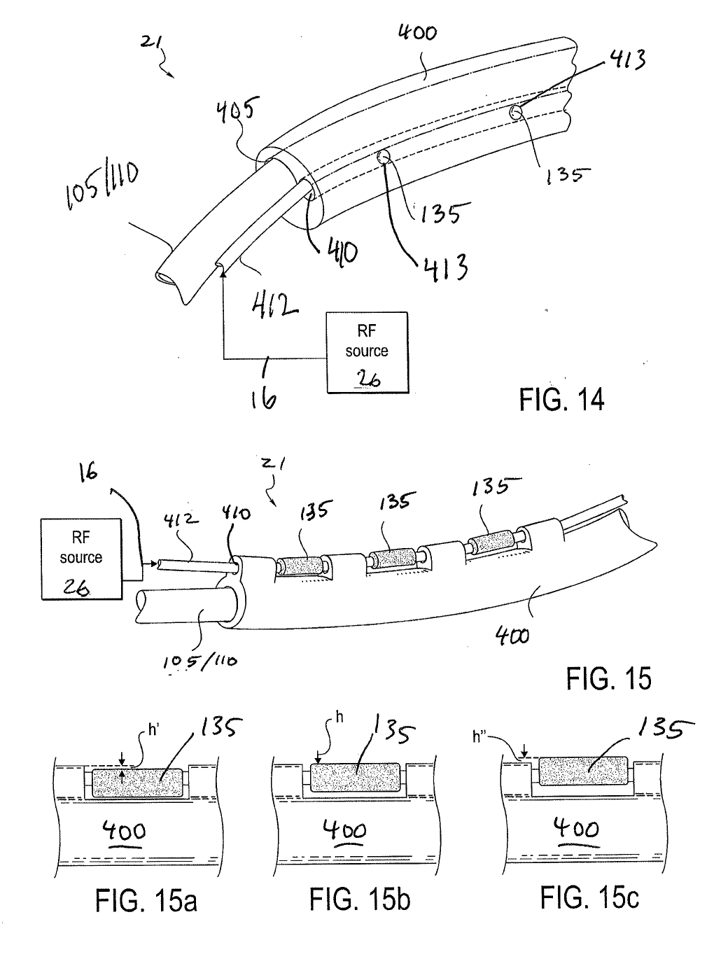

While the various alternative therapy systems described herein may be employed in an intraluminal or transluminal therapy mode, an exemplary intraluminal therapy will be described with regard to a treatment for the left and right renal arteries alone or in combination. Since neuromodulation of a left and/or right renal plexus RP may be achieved in accordance with the present technology through intraluminal, in this specific example, intravascular access, properties and characteristics of the specific lumen may impose limitations, constraints upon and/or inform the design of apparatus, systems, and methods for achieving such renal neuromodulation via the renal vasculature. However, as the description that follows will make clear, the spiral support frame described herein may advantageously be position into a wide variety of luminal sizes and shapes and still maintain appropriate apposition and provide a stable platform for therapy delivery. In some embodiments, the actual designs of a support frame and other properties and characteristics may vary across the patient population and/or within a specific patient across time, as well as in response to disease states, such as hypertension, chronic kidney disease, vascular disease, end-stage renal disease, insulin resistance, diabetes, metabolic syndrome, etc. These properties and characteristics, as explained herein, may have bearing on the efficacy of the procedure and the specific design of the intravascular device, specifically as to the selection of multi-mode therapy devices and other features or configurations of the devices described herein. Properties of interest that provide advantageous characteristics of the delivery device include, for example, material/mechanical, spatial, fluid dynamic/hemodynamic and/or thermodynamic properties of the spiral support frame. As will be appreciated by the various support frame shapes (see FIGS. 12A-12E), and the use of one or more conduits of a multi-lumen device (see FIGS. 14-17B) to provide multiple treatment modalities while maintaining the support frame along the vessel wall in use.

The multiple lumen sheath 400 may be provided in a number of different embodiments for providing one or more of a support frame lumen 405, a therapy element lumen 410, a drug perfusion lumen 415, a fixation element lumen 420, or an additional lumen 425 in any combination or orientation or spatial relationship as illustrated in FIGS. 14, 15, 15A, 15B, 15C, 16A-16F and 17B. Additionally, one or more apertures appropriate to the respective lumen may also be provided in the sidewall of the respective lumen. For example, a therapy element lumen 410 may include one or more apertures 413 sized for a particular therapy element 135. A perfusion lumen 410 may include one or more apertures 417. A fixation element lumen 420 may include apertures 422 for one or more fixation elements 805.

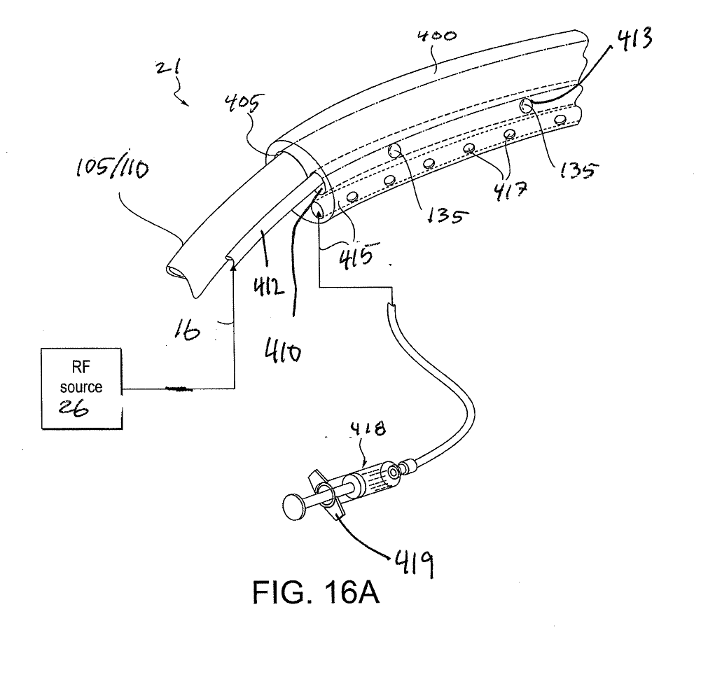

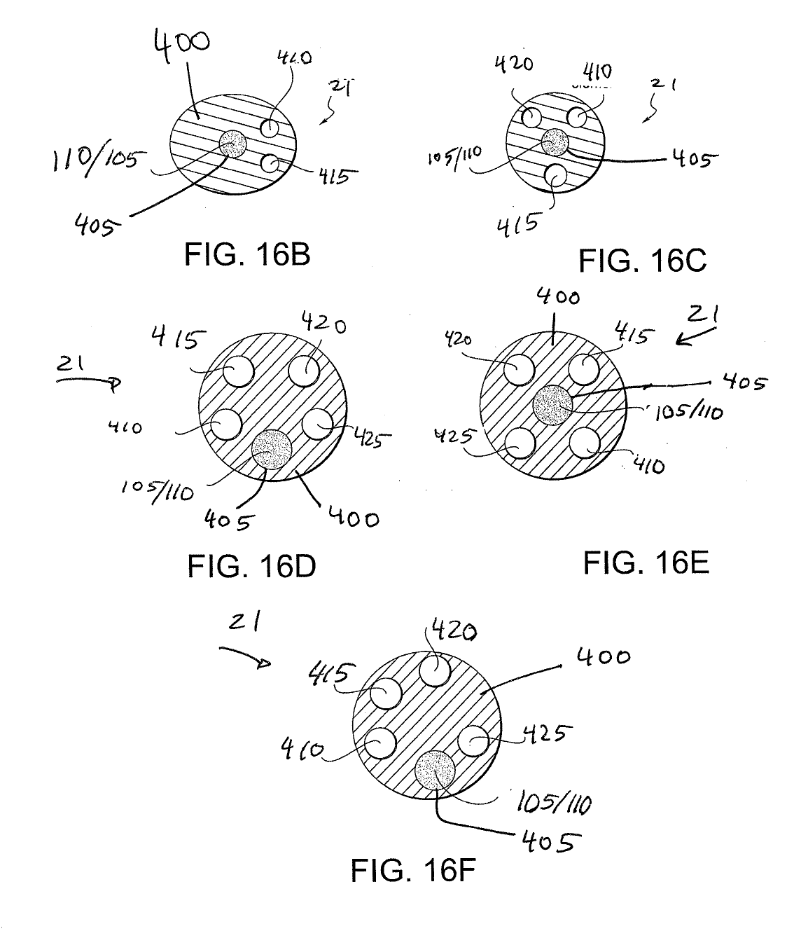

FIG. 16A illustrates a multiple lumen sheath 400 embodiment of a delivery device 21 configured for delivery of both electrode and drug therapy. The lumen 405 is configured to receive a support frame element 105, 110. The lumen 410 is configured to receive the electrical connections 412 to therapy elements 135 positioned within appropriately sized and positioned apertures 413. The lumen 415 is configured to provide communication between an external or internal drug reservoir or pump and one or more apertures 417 in communication with the lumen 415. In the illustrated embodiment, there is a pharmacologically active compound or agent 418 within a syringe 419. Operation of the syringe delivers the agent 419 to the intraluminal treatment site via the one or more apertures 417.

Returning to the specific example of the renal vasculature, a catheter may be advanced percutaneously into either the left or right renal artery via a minimally invasive intravascular path. However, while a minimally invasive renal arterial access may be challenging for conventional devices, embodiments of the present invention have a collapsible support frame that permits access to the renal arteries. While renal arteries are often extremely tortuous, may be of relatively small diameter, and/or may be of relatively short length the ability of the support frame described herein may expand to accommodate a variety of luminal sizes. FIGS. 10A, 10B and 10C illustrate well how the sliding crossover of the support frame permits the use of a device that can size to fit within a range of lumen dimensions.

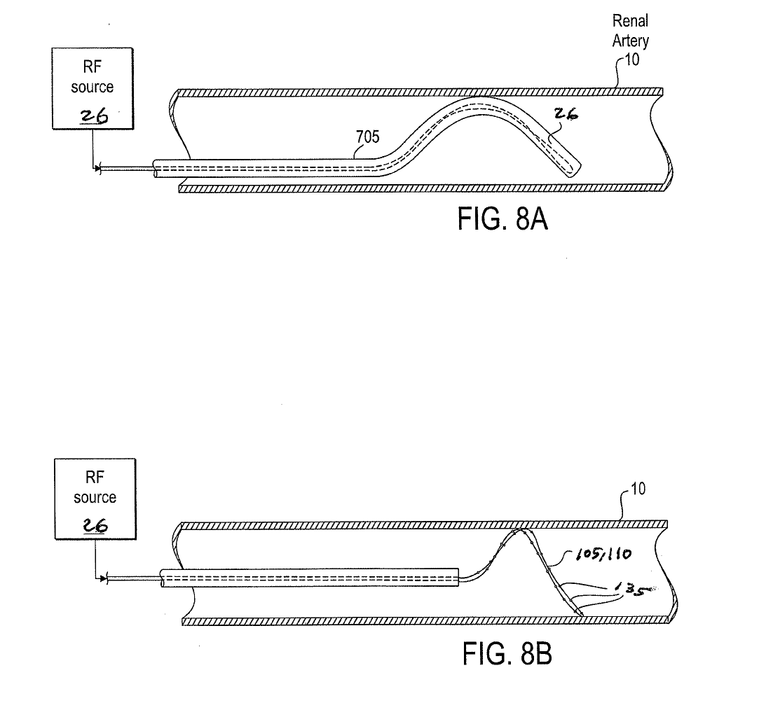

Furthermore, renal arterial atherosclerosis is common in many patients, particularly those with cardiovascular disease. Renal arterial anatomy also may vary significantly from patient to patient, which further complicates minimally invasive access. Significant inter-patient variation may be seen, for example, in relative tortuosity, diameter, length, and/or atherosclerotic plaque burden, as well as in the take-off angle at which a renal artery branches from the aorta. Exemplary apparatus, systems and methods for achieving renal neuromodulation via intravascular access as described herein may account for these and other aspects of renal arterial anatomy and its variation by virtue of the unique shape of the stowed device during delivery as best seen in FIG. 8A. The curved shape of the delivery catheter allows the device to be steered through the vasculature by rotating the catheter shaft. Additionally, the reversible deployment of the support frame permits partial deployment as shown in FIG. 8B to evaluation placement or position before therapy delivery. As a result of the unique nature of the reversible support frame, the various embodiments described herein may be utilized for delivery of intraluminal and transluminal therapy across patient populations. The designs disclosed herein are particularly suited for when minimally invasively accessing a renal artery.

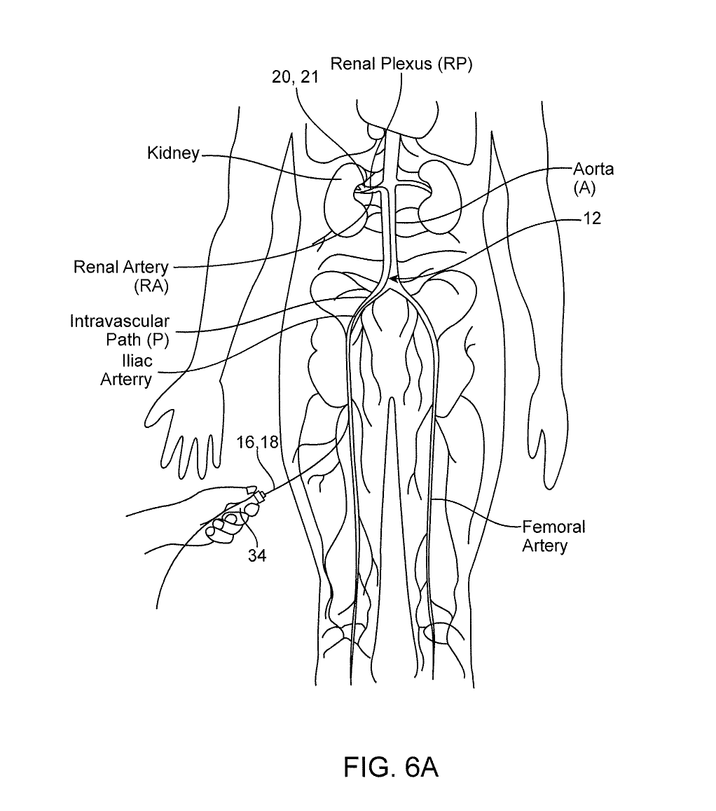

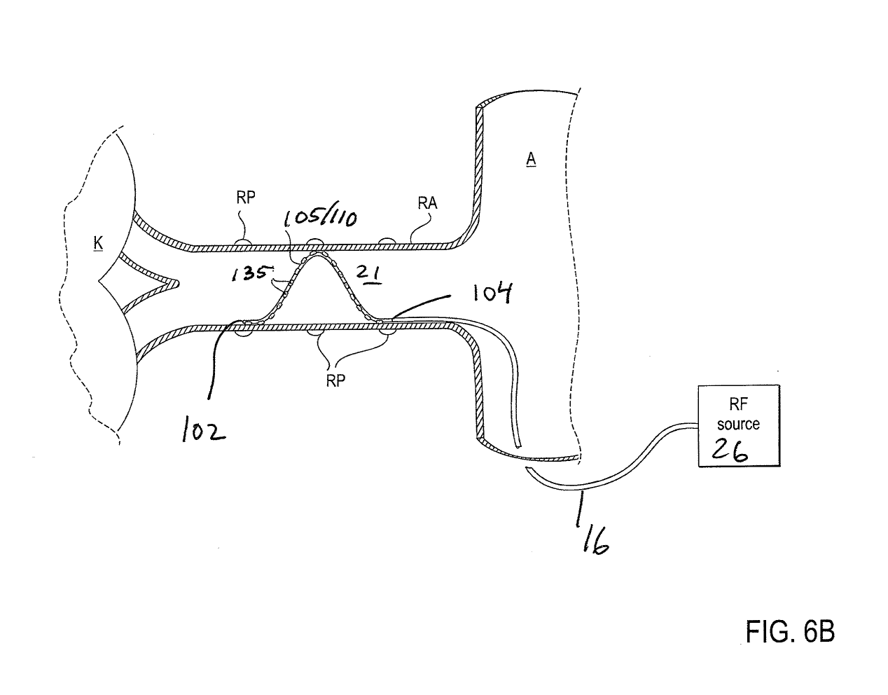

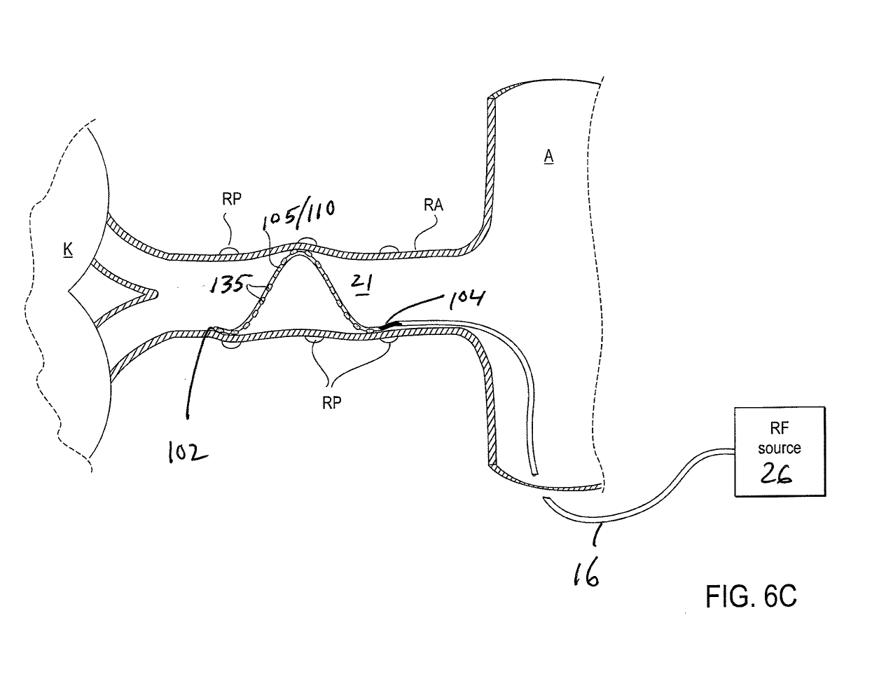

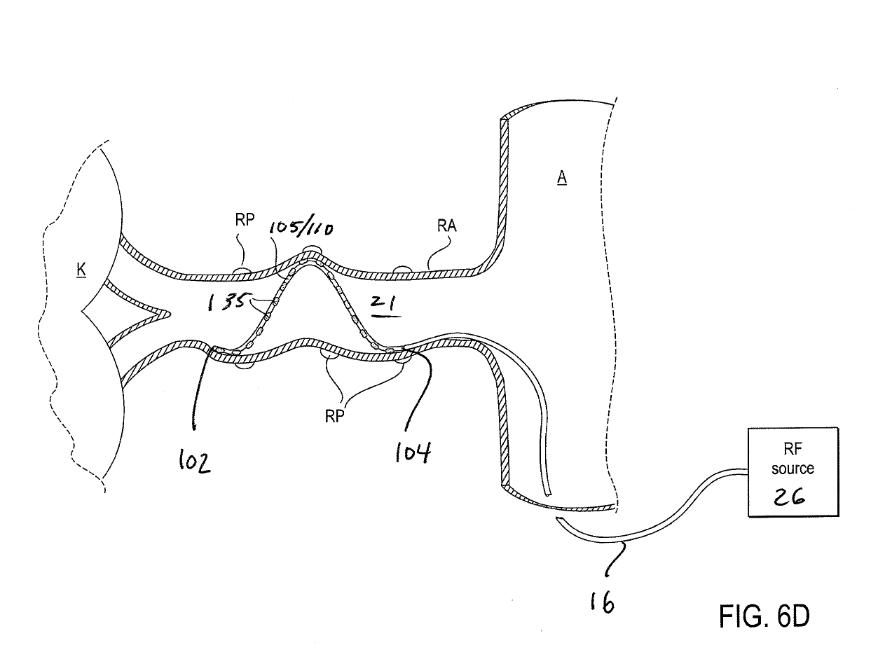

In addition to complicating renal arterial access, specifics of the renal anatomy also complicate establishment of stable contact between neuromodulatory apparatus and a luminal surface or wall of a renal artery. When the neuromodulatory apparatus includes an energy delivery element, such as an electrode, consistent positioning and appropriate contact force applied by the energy delivery element to the vessel wall are important for predictability. Sufficient therapy element apposition may be provided in a variety of ways. In one aspect, therapy device to lumen wall apposition is provided by altering the cross section of the support frame (see FIGS. 12A-12D), the outward force generated by a particular frame shape, alone or in combination with the size, shape, number or position of the therapy elements along the support frame. In addition or alternatively, the outward force of the support frame itself may be used of generate variable lumen wall forces as shown by FIGS. 6B, 6C and 6D.

In addition or alternatively, the outward force of an individual therapy element may itself may be used of generate variable lumen wall forces as shown by the positioning of therapy elements in FIGS. 15, 15A, 15B and 15C. In one aspect, there is provided a delivery device used to adjust the outward forces generated by the spiral support frame. The device may be used to temporarily hold the shape and size of the frame during delivery of therapy. Additionally or optionally, the delivery device and the support frame may be adapted and configured for an indexed or stepwise delivery of the support frame into different shapes (i.e., same size loops or different size loops), lengths and/or degree of apposition force applied against tissue in a delivery or implantation location. In still another aspect, the relative position or size of a therapy element may be adjusted or selected to adjust the contact or apposition of the therapy element with the lumen wall. FIGS. 15a, 15b and 15c illustrate an exemplary variation of this concept. FIG. 15a illustrates a therapy element having an outer surface that is recessed below the adjacent outer surface of the support frame. In this illustrative embodiment, the outer surface of the therapy element is recessed by a height h′ below the surface of the support frame. FIG. 15b illustrates a therapy element having an outer surface that is not recessed below the adjacent outer surface of the support frame but is at the same height. In this illustrative embodiment, the outer surface of the therapy element is positioned at the same height h as the surface of the support frame. FIG. 15c illustrates a therapy element having an outer surface that is positioned above the adjacent outer surface of the support frame. In this illustrative embodiment, the outer surface of the therapy element is extending above the adjacent support frame by a height h″. In one aspect, the size upon delivery, during therapy or as an implanted device is based on a setting or an index on the handle of the delivery device. In one aspect, there is provided an indexed delivery marking or markings or scale to aid the user in determining the size of the support frame.

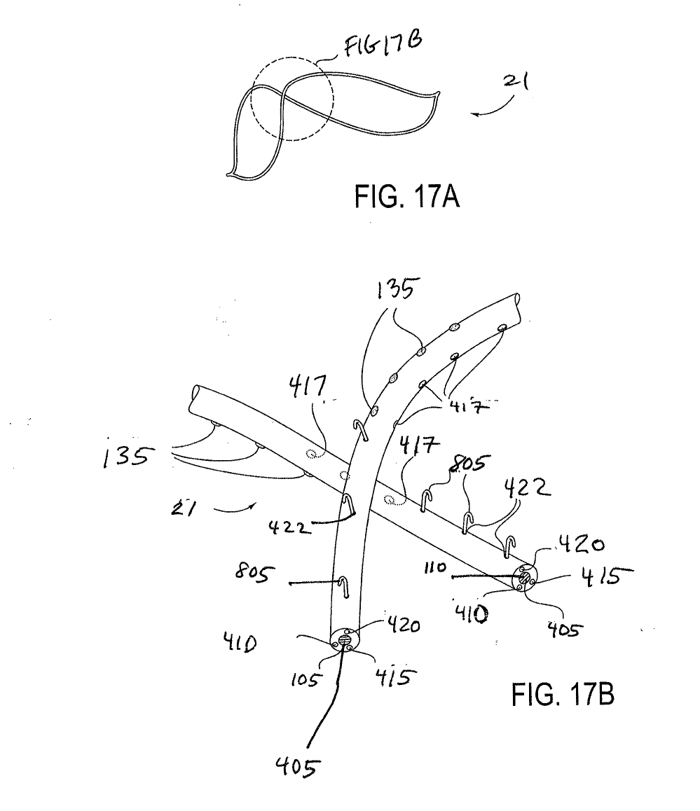

Moreover, in some intraluminal or transluminal treatment locations, establishing consistent therapy element contact is complicated by patient movement, respiration, and/or the cardiac cycle. This is especially true in the renal vasculature because these same factors may cause significant movement of the renal artery relative to the aorta, and the cardiac cycle may transiently distend the renal artery (i.e., cause the wall of the artery to pulse). Stability of the therapy devices described herein during vessel movement may, for example, utilize one or more penetrating therapy elements (see FIG. 12H) that may penetrate into or through a luminal wall as illustrated and described in U.S. Pub. No. US-2013-0053792-A1 to Fischell et al., which is herein incorporated by reference in its entirety. Additionally or alternatively, one or more fixed or retractable fixation elements (see FIG. 17B) may be provided for device stabilization to counter the forces described above.

Even after accessing a renal artery and facilitating stable contact between neuromodulatory apparatus and a luminal surface of the artery, nerves in and around the adventia of the artery should be safely modulated via the neuromodulatory apparatus. Effectively applying thermal treatment from within a renal artery is non-trivial given the potential clinical complications associated with such treatment. For example, the intima and media of the renal artery are highly vulnerable to thermal injury. As discussed in greater detail below, the intima-media thickness separating the vessel lumen from its adventitia means that target renal nerves may be multiple millimeters distant from the luminal surface of the artery. Sufficient energy should be delivered to or heat removed from the target renal nerves to modulate the target renal nerves without excessively cooling or heating the vessel wall to the extent that the wall is frozen, desiccated, or otherwise potentially affected to an undesirable extent. A potential clinical complication associated with excessive heating is thrombus formation from coagulating blood flowing through the artery. Given that this thrombus may cause a kidney infarct, thereby causing irreversible damage to the kidney, thermal treatment from within the renal artery should be applied carefully. Accordingly, the complex fluid mechanics and thermodynamic conditions present in the renal artery during treatment, particularly those that may impact heat transfer dynamics at the treatment site, may be important in applying energy (e.g., heating thermal energy) and/or removing heat from the tissue (e.g., cooling thermal conditions) from within the renal artery.

The neuromodulatory apparatus should also be configured to allow for adjustable positioning and repositioning of the energy delivery element within the renal artery since location of treatment may also impact clinical efficacy. For example, it may be tempting to apply a full circumferential treatment from within the renal artery given that the renal nerves may be spaced circumferentially around a renal artery. In some situations, full-circle lesion likely resulting from a continuous circumferential treatment may be potentially related to renal artery stenosis. Therefore, the formation of more complex lesions along a longitudinal dimension of the renal artery via the spiral shaped supports and therapy elements and other structures described herein and/or repositioning of the neuromodulatory apparatus to multiple treatment locations may be desirable. It should be noted, that a benefit of the reversible, collapsible spiral frame is relative ease of multiple placements via rotation of the frame for creating a desired therapeutic ablation pattern.

Additionally, variable positioning and repositioning of the spiral framed neuromodulatory apparatus may prove to be useful in circumstances where the renal artery is particularly tortuous or where there are proximal branch vessels off the renal artery main vessel. It is also believed that the delivery and manipulation of the spiral frame devices herein in a renal artery would not produce mechanical injury on the renal artery. Motion of the device in an artery, for example by inserting, manipulating, negotiating bends and so forth, may not contribute appreciably to dissection, perforation, denuding intima, or disrupting the interior elastic lamina.

Blood flow through a renal artery may be temporarily occluded for a short time with minimal or no complications. However, occlusion for a significant amount of time should be avoided because to prevent injury to the kidney such as ischemia. Because of the advantageous against the wall frame design, embodiments herein avoid occlusion all together.

Based on the above described challenges of (1) renal artery intervention, (2) consistent and stable placement of the treatment element against the vessel wall, (3) effective application of treatment across the vessel wall, (4) positioning and potentially repositioning the treatment apparatus to allow for multiple treatment locations, and (5) avoiding or limiting duration of blood flow occlusion, various independent and dependent properties of the renal vasculature that may be of interest include, for example, (a) vessel diameter, vessel length, intima-media thickness, coefficient of friction, and tortuosity; (b) distensibility, stiffness and modulus of elasticity of the vessel wall; (c) peak systolic, end-diastolic blood flow velocity, as well as the mean systolic-diastolic peak blood flow velocity, and mean/max volumetric blood flow rate; (d) specific heat capacity of blood and/or of the vessel wall, thermal conductivity of blood and/or of the vessel wall, and/or thermal convectivity of blood flow past a vessel wall treatment site and/or radiative heat transfer; (e) renal artery motion relative to the aorta induced by respiration, patient movement, and/or blood flow pulsatility: and (f) as well as the take-off angle of a renal artery relative to the aorta. These properties will be discussed in greater detail with respect to the renal arteries. However, dependent on the apparatus, systems and methods utilized to achieve renal neuromodulation, such properties of the renal arteries, also may guide and/or constrain design characteristics.

As noted above, an apparatus positioned within a renal artery should conform to the geometry of the artery. Renal artery vessel diameter, D_{RA}, typically is in a range of about 2-10 mm, with most of the patient population having a D_{RA} of about 4 mm to about 8 mm and an average of about 6 mm. Renal artery vessel length, L_{RA}, between its ostium at the aorta/renal artery juncture and its distal branchings, generally is in a range of about 5-70 mm, and a significant portion of the patient population is in a range of about 20-50 mm. Since the target renal plexus is embedded within the adventitia of the renal artery, the composite Intima-Media Thickness, IMT, (i.e., the radial outward distance from the artery's luminal surface to the adventitia containing target neural structures) also is notable and generally is in a range of about 0.5-2.5 mm, with an average of about 1.5 mm. Although a certain depth of treatment is important to reach the target neural fibers, the treatment should not be too deep (e.g., >5 mm from inner wall of the renal artery) to avoid non-target tissue and anatomical structures such as the renal vein.

An additional property of the renal artery that may be of interest is the degree of renal motion relative to the aorta, induced by respiration and/or blood flow pulsatility. A patient's kidney, which located at the distal end of the renal artery, may move as much as 4″ cranially with respiratory excursion. This may impart significant motion to the renal artery connecting the aorta and the kidney, thereby requiring from the neuromodulatory apparatus a unique balance of stiffness and flexibility to maintain contact between the thermal treatment element and the vessel wall during cycles of respiration. Furthermore, the take-off angle between the renal artery and the aorta may vary significantly between patients, and also may vary dynamically within a patient, e.g., due to kidney motion. The take-off angle generally may be in a range of about 30°-135°.

IV. Selected Embodiments of Intraluminal Neuromodulation Systems and Devices

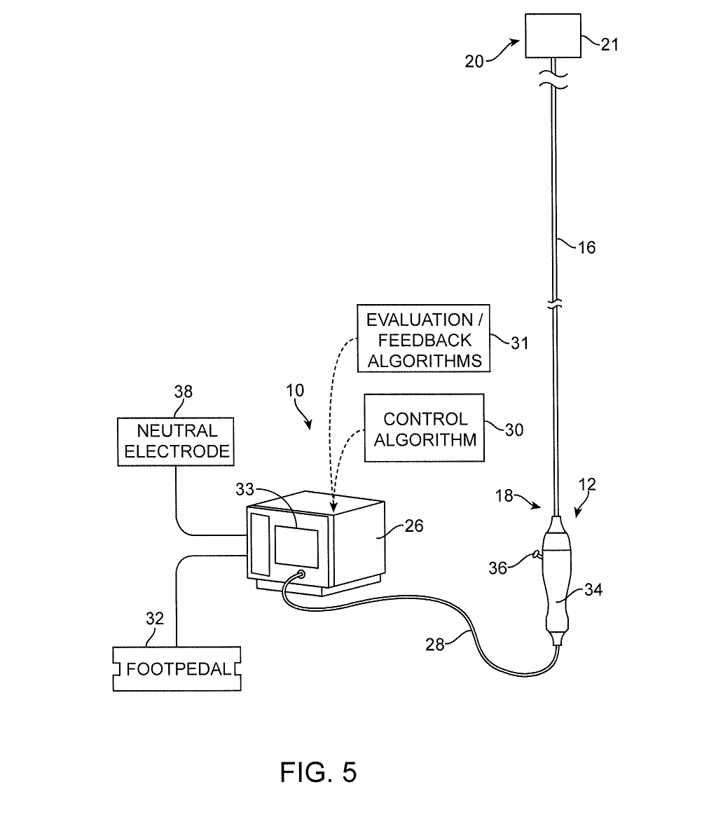

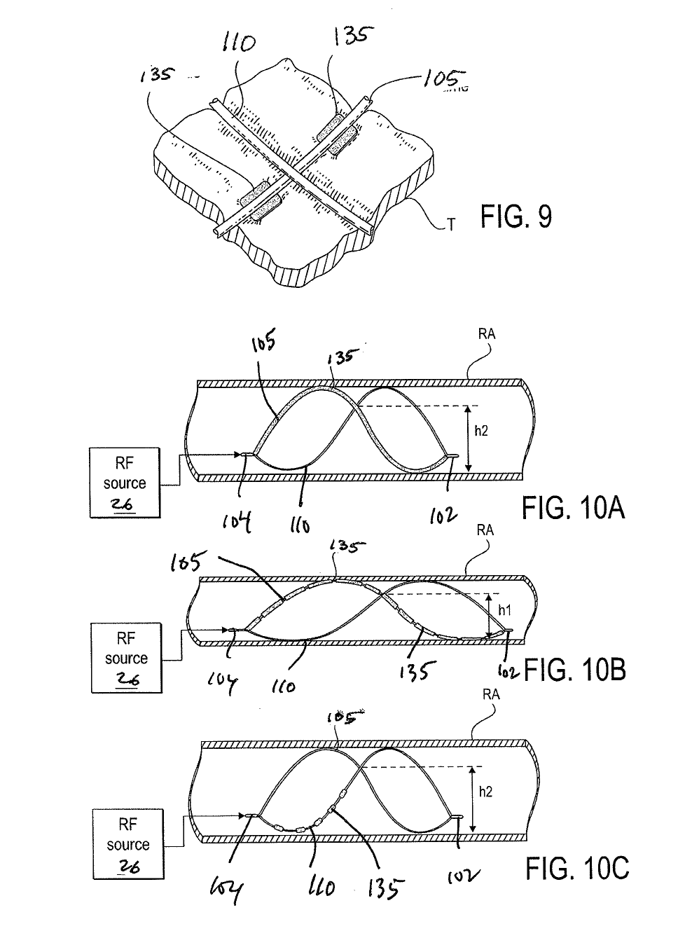

FIG. 5 illustrates an intraluminal neuromodulation system 10 configured in accordance with an embodiment of the present technology. The system 10, for example, may be used to perform therapeutically-effective renal neuromodulation on a patient. The system 10 includes an intravascular treatment device 12 operably coupled to a suitable energy source or console 26 (e.g., a radiofrequency energy generator, a cryotherapy console, ultrasound or other therapeutic energy modality). In the embodiment shown in FIG. 5, the treatment device 12 (e.g., a catheter) includes an elongated shaft 16 having a proximal portion 18, a handle 34 at a proximal region of the proximal portion 18, and a distal portion 20 extending distally relative to the proximal portion 18. The treatment device 12 further includes a support frame neuromodulation assembly or treatment section 21 at the distal portion 20 of the shaft 16. The neuromodulation assembly 21 can include one or more electrodes or energy-delivery elements, a cryotherapeutic cooling assembly and/or a nerve monitoring device configured to be delivered to a renal blood vessel (e.g., a renal artery) in a low-profile configuration using a support frame described herein (see, e.g., illustrative embodiments of FIGS. 6B, 6C, 6D and 8A-18C).

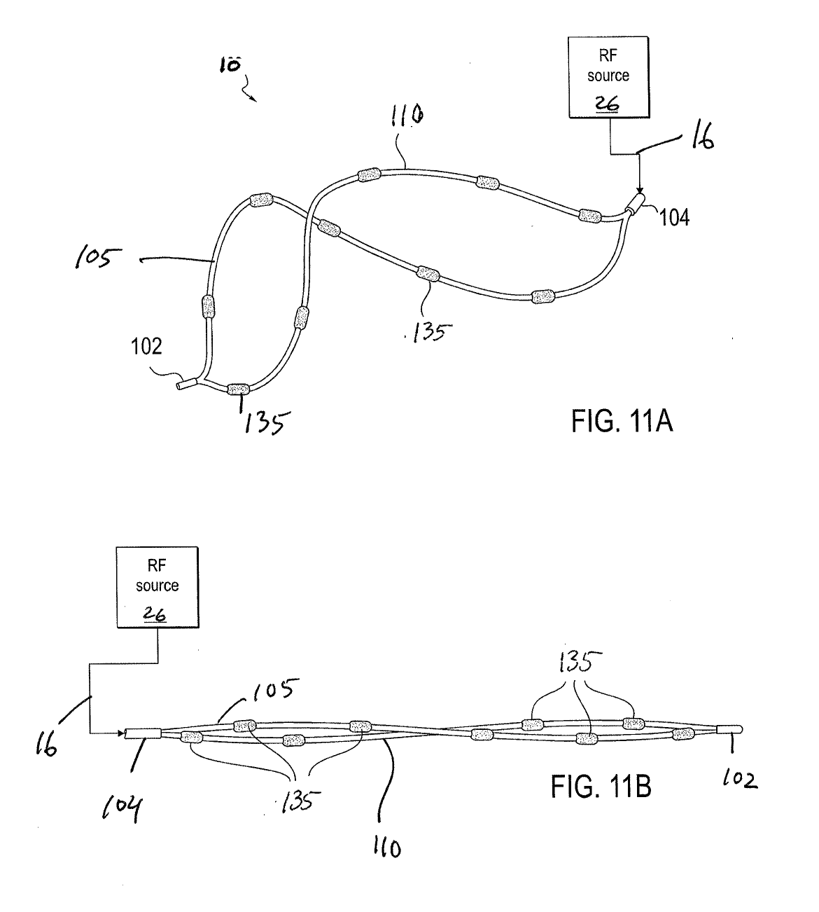

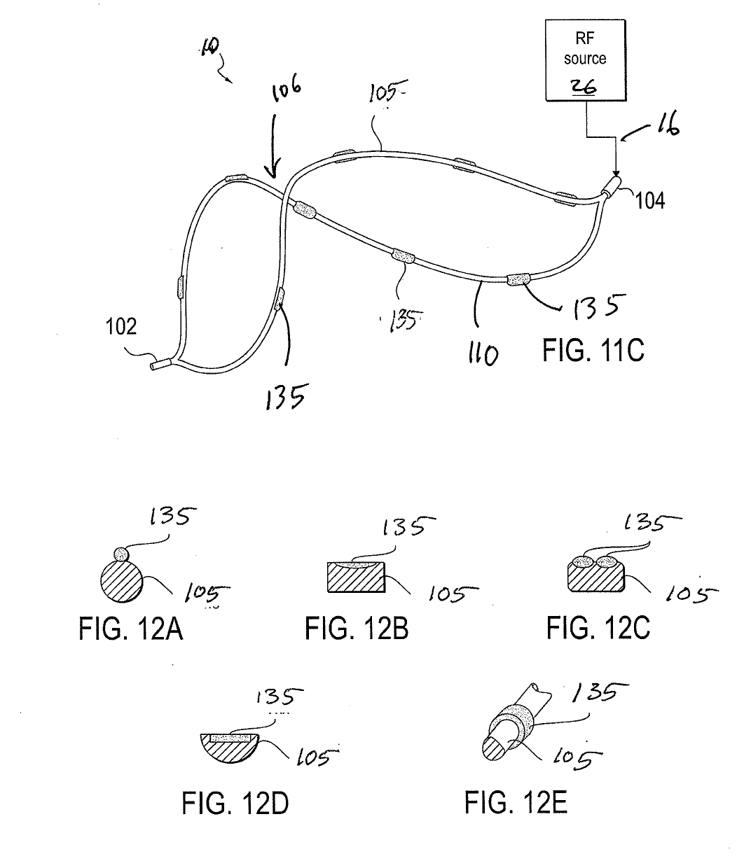

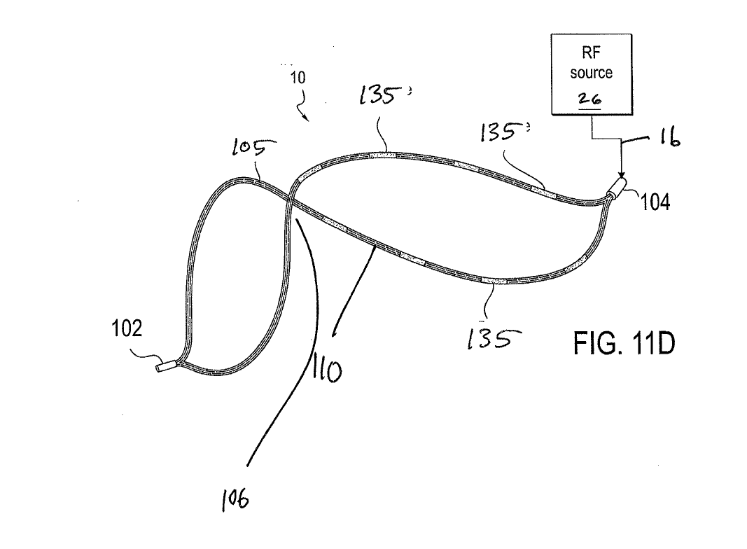

Upon delivery to a target treatment site within a renal blood vessel (RA), the neuromodulation assembly 21 can be further configured to be deployed into a treatment state or arrangement for delivering energy at the treatment site and providing therapeutically-effective electrically-induced and/or thermally-induced renal neuromodulation using one or more therapy elements borne by the support frame. In some embodiments, the neuromodulation assembly 21 may be placed or transformed from a stowed configuration (FIGS. 8A and 11B) into the deployed state (FIGS. 6B, 6C, 6D, 8B, 10A, 10B or 10C) or arrangement via remote actuation, e.g., via an actuator 36, such as a knob, pin, or lever carried by the handle 34. In other embodiments, however, the neuromodulation assembly 21 may be transformed between the delivery and deployed states using other suitable mechanisms or techniques such as pulling back a sheath or advancing the therapy device out of a sheath. The proximal end of the neuromodulation assembly 21 can be carried by or affixed to the distal portion 20 of the elongated shaft 16. A distal end of the neuromodulation assembly 21 may terminate with, for example, an atraumatic rounded tip or cap. Alternatively, the distal end of the neuromodulation assembly 21 may be configured to engage another element of the system 10 or treatment device 12. For example, the distal end of the neuromodulation assembly 21 may define a passageway for engaging a guide wire (not shown) for delivery of the treatment device using over-the-wire (“OTW”) or rapid exchange (“RX”) techniques.

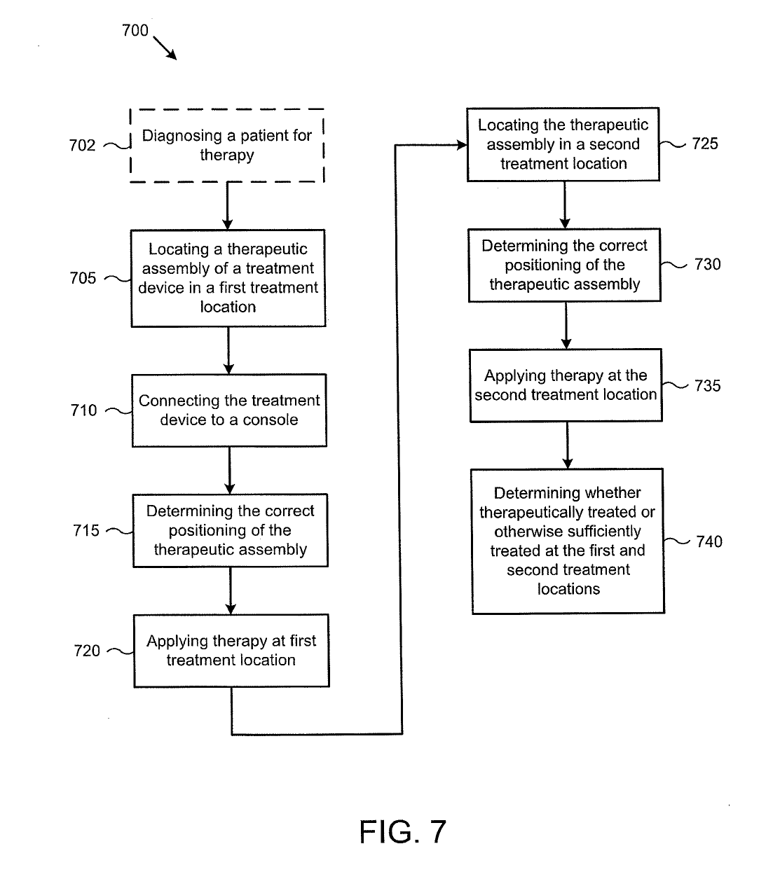

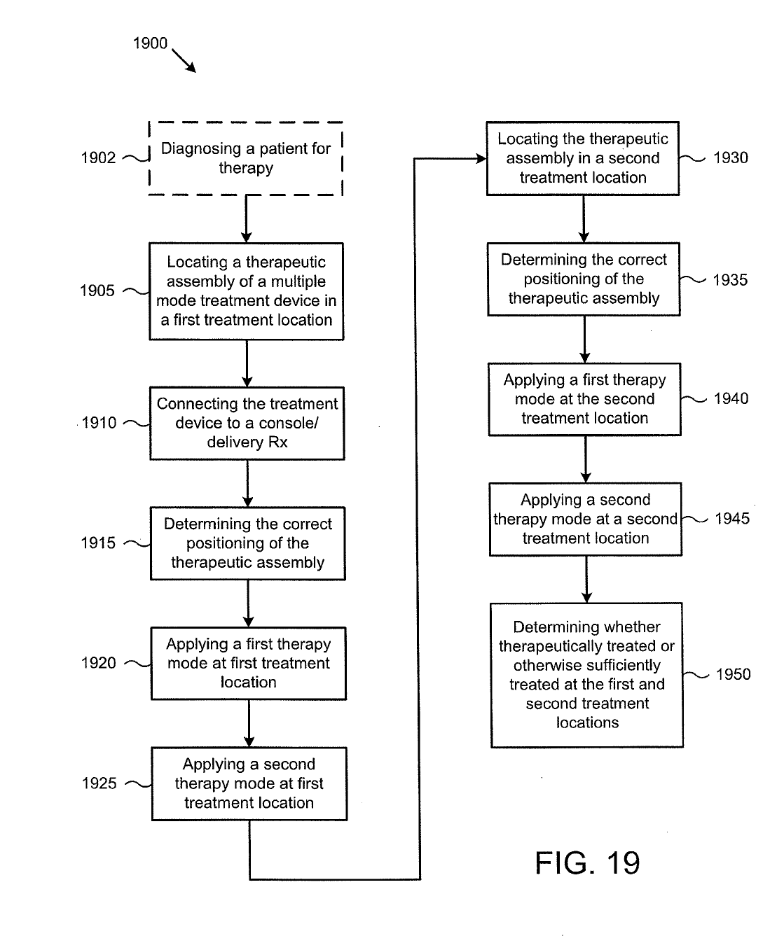

The energy source or console 26 can be configured to generate a selected form and magnitude of energy for delivery to the target treatment site via the one or more therapeutic elements within the neuromodulation assembly 21. A control mechanism, such as a switch or foot pedal 32, may be connected (e.g., pneumatically connected or electrically connected) to the energy source or console 26 to allow an operator to initiate, terminate and, optionally, adjust various operational characteristics of the energy source or console 26, including, but not limited to, power delivery. FIG. 5 may also include other auxiliary components depending upon the needs of the energy model embodiment in a particular energy source 26/spiral support frame treatment device 21 configuration. In the illustrative embodiment, the system 10 includes a neutral electrode 38—that may not be required for other types of system. The system 10 may also include a remote control device (not shown) that can be positioned in a sterile field and operably coupled to the neuromodulation assembly 21. The remote control device can be configured to allow for selective activation of the neuromodulation assembly 21. In other embodiments, the remote control device may be built into the handle assembly 34. The energy source 26 can be configured to deliver the treatment energy via a computer controlled or software implemented, automated or semi-automated control algorithm 30 and/or under the control of the clinician. In addition, the energy source 26 may include one or more evaluation or feedback algorithms 31 to provide feedback to the clinician or the algorithm 30 before, during, and/or after therapy (FIGS. 7 and 19).

The energy source 26 can further include a device or monitor that may include processing circuitry, such as a microprocessor, and a display 33. The processing circuitry may be configured to execute stored instructions relating to the control algorithm 30. The energy source 26 may be configured to communicate with the treatment device 12 (e.g., via a cable 28) to control the neuromodulation assembly and/or to send signals to or receive signals from the nerve monitoring device. The display 33 may be configured to provide indications of power levels or sensor data, such as audio, visual or other indications, or may be configured to communicate information to another device. For example, the console 26 may also be configured to be operably coupled to a catheter lab screen or system for displaying treatment information, such as nerve activity before and/or after treatment along with real time imaging information such as intravascular ultrasound carried by or positioned along with the therapy device.

FIG. 6A illustrates modulating renal nerves with an embodiment of the system 10. The treatment device 12 provides access to the renal plexus RP through an intravascular path, such as a percutaneous access site in the femoral (illustrated), brachial, radial, or axillary artery to a targeted treatment site within a respective renal artery RA. As illustrated, a section of the proximal portion 18 of the shaft 16 is exposed externally of the patient. By manipulating the proximal portion 18 of the shaft 16 from outside the intravascular path P, the clinician may advance the shaft 16 through the sometimes tortuous intravascular path P and remotely manipulate the distal portion 20 of the shaft 16. Image guidance, e.g., computed tomography (CT), fluoroscopy, intravascular ultrasound (IVUS), optical coherence tomography (OCT), or another suitable guidance modality, or combinations thereof, may be used to aid the clinician's manipulation. Further, in some embodiments, image guidance components (e.g., IVUS, OCT) may be incorporated into the treatment device 12. Additionally or alternatively, a therapy device described herein may be modified to have enhanced echogenic characteristics to assist in ultrasound assisted positioning the device for therapy within a lumen. The ultrasound assisted placement may be provided by external or intraluminal ultrasound devices. Additional details for echogenic enhancement as well as exemplary intraluminal ultrasound devices and systems are provided in U.S. Prov. Pat. App. No. 61/785,955, filed Mar. 14, 2013, titled “ENDOLUMINAL FILTER HAVING ENHANCED ECHOGENIC PROPERTIES,” which is incorporated herein by reference in its entirety.

After the neuromodulation assembly 21 is adequately positioned in the renal artery RA, it can be radially expanded or otherwise deployed using the handle 34 or other suitable control mechanism until the neuromodulation assembly is positioned at its target site and in stable contact with the inner wall of the renal artery RA by outward forces of the spiral support frame. The purposeful application of energy from the neuromodulation assembly can then be applied to tissue to induce one or more desired neuromodulating effects on localized regions of the renal artery RA and adjacent regions of the renal plexus RP, which lay intimately within, adjacent to, or in close proximity to the adventitia of the renal artery RA. The neuromodulating effects may include denervation, thermal ablation, and non-ablative thermal alteration or damage (e.g., via sustained heating and/or resistive heating). The purposeful application of the energy may achieve neuromodulation along all or at least a portion of the renal plexus RP.

FIGS. 6B, 6C and 6D illustrate section views of a treatment device 21 in position to delivery therapy in a renal artery with varying levels of apposition to the vessel wall. FIG. 6B illustrates a treatment device 21 where the size, shape, radial forces and/or development of the support frame 105, 110 places the therapy elements 135 in contact with the vessel wall RA without distention. FIG. 6C illustrates a treatment device 21 where the size, shape, radial forces and/or deployment of the support frame 105, 110 places the therapy elements 135 into the luminal wall RA so as to cause some distention or mild distention of the wall RA as shown. FIG. 6D illustrate a treatment device 21 where the size, shape, radial forces and/or deployment of the support frame 105, 110 places the therapy elements 135 into the luminal wall RA and thereby distend the wall to a greater degree than in FIG. 6C. Various alternative configurations of the support frame 105, 110 may be utilized along with manipulation of the spacing between proximal and distal ends 104, 102 to vary the shape and distention forces imparted by the support frame elements 105, 110.

Further details of the construction, operation and properties of the support frame and other aspects of the spiral frame delivery devices are provided in U.S. patent application Ser. No. 13/553,335, filed Jul. 19, 2012, titled “ENDOLUMINAL FILTER WITH FIXATION,” Publication No. US-2013-0012981-A1 and International Pat. App. No. PCT/US2008/088606, filed Dec. 31, 2008, titled “ENDOLUMINAL FILTER WITH FIXATION,” Pub. No. WO 2009/088905, each of which is incorporated herein by reference in its entirety.

As mentioned previously, the therapy elements, devices and methods of use for system 10 and source 26 disclosed herein may provide intraluminal or transluminal stimulation, modulation and/or ablative therapy to a patient using one or a variety of suitable energy modalities. Accordingly, the system 10 and source 26 may be adapted and configured along with appropriate alterations to the therapy elements and support frame for the delivery of: including RF energy, microwave energy, laser, optical energy, ultrasound, HIFU, magnetic energy, direct heat, cryotherapy, or a combination thereof with or without penetrating therapy elements or, with or without stabilizing elements. In one aspect, various aspects of the therapeutic delivery device may be modified, adapted and configured for the delivery of microwave energy such as described in U.S. patent application Ser. No. 13/281,244, filed Oct. 25, 2011, titled “MICROWAVE CATHETER APPARATUSES, SYSTEMS, AND METHODS FOR RENAL NEUROMODULATION,” Pub. No. US-2012-0116486-A1, incorporated herein by reference in its entirety. The therapy device 21 may be configured as a multi-lumen sheath 400 having a plurality of lumens to support a variety of independent therapy modalities on a single support frame device. FIG. 14 illustrates a portion of a multi-lumen sheath 400 configured for use as a support frame device 21. In this embodiment, there are two lumens 405 and 410 provided within the multi-lumen sheath 400. The lumen 405 is sized to receive the support frames 105, 110. The lumen 410 is sized and configured to receive the therapy element 135 along with the associated connections to the energy source 26. In the embodiment of FIG. 14, the therapy element 135 is an electrode and the conduit 410 includes an appropriate electrical connection 412 depending upon the configuration being used. Multiple lumen embodiments may be configured into a variety of different cross section shapes such as oval (SEE FIGS. 14, 16B) or figure 8 (see FIGS. 15, 15A, 15B, 15C where the smaller electrode conduit 410 is adjacent to the larger support frame conduit 405 giving the therapy device a figure-8 shape) or circular (see FIGS. 16C-16F).

Alternatively or in addition to these techniques, the methods may utilize one or more non-ablative neuromodulatory techniques. For example, the methods may utilize non-ablative SNS denervation by removal of target nerves, injection of target nerves with a destructive drug or pharmaceutical compound, or treatment of the target nerves with non-ablative energy modalities. In certain embodiments, the amount of reduction of the sympathetic nerve activity may vary depending on the specific technique being used. In still further embodiments, a pharmacologically inactive perfusion fluid is provide prior to, during or after the use of one or more therapy elements to provide intraluminal or transluminal stimulation, modulation and/or ablative therapy. In a still further example, a method may utilize non-ablative SNS denervation by removal of target nerves, injection of target nerves with a destructive drug or pharmaceutical compound, or treatment of the target nerves with non-ablative energy.

In certain embodiments, a neuromodulation or neurostimulation device for use in the methods disclosed herein may combine two or more energy modalities alone or in combination with one or more perfusion compositions (see FIGS. 17B and 19). For example, the device may include both a hyperthermic source of ablative energy and a hypothermic source, making it capable of, for example, performing both RF ablation and cryoablation. In still other various alternative configurations, embodiments of the therapeutic devices described herein may be adapted or configured according to one or more of the devices, components or configurations described in U.S. patent application Ser. No. 13/691,594, filed Nov. 30, 2012, titled “RENAL NEUROMODULATION METHODS AND DEVICES FOR TREATMENT OF POLYCYSTIC KIDNEY DISEASE,” Publ. No. US-2013-0144251-A1, which is incorporated herein by reference in its entirety. Additionally or alternatively, the treatment device may be configured to carry out one or more non-ablative neuromodulatory techniques. For example, the device may comprise a means for diffusing a drug or pharmaceutical compound at the target treatment area using one or more of the apertures of a delivery conduit. FIG. 16A illustrates a multiple lumen sheath embodiment of a delivery device 21 configured for delivery of both electrode and drug therapy. The lumen 405 is configured to receive a support frame element 105, 110. The lumen 410 is configured to receive the electrical connections 412 to therapy elements 135. The lumen 415 is configured to provide communication between an external or internal drug reservoir or pump and one or more apertures 417 in communication with the lumen 415. In the illustrated embodiment, there is a pharmacologically active compound or agent 418 within a syringe 419. Operation of the syringe delivers the agent 419 to the intraluminal treatment site via the one or more apertures 417. In one aspect, one or more apertures 417 in a delivery conduit 415 may have flat walls, divergent walls or convergent walls. By adjusting the aperture of a delivery conduit, a perfusion embodiment may be adapted to provide spray diffusion or mixing or directionality of the perfusion or fluid injection flow. A series of exemplary straight (non-divergent) aperture walls is illustrated in FIG. 16A. In an exemplary aperture with a divergent aperture shape the diameter of the opening on the inner conduit wall is smaller than the aperture opening on the outer conduit wall. In an exemplary aperture with a convergent aperture shape the diameter of the opening on the inner conduit wall is larger than the aperture opening on the outer conduit wall. Additionally or alternatively, the aperture used for a perfusion port may be at an angle from the main perfusion conduit. In still other various embodiments, embodiments of the therapeutic devices described herein may be adapted or configured according to one or more of the devices, components or configurations described in U.S. patent application Ser. No. 13/278,081, filed Oct. 20, 2011, titled “CATHETER APPARATUSES HAVING EXPANDABLE MESH STRUCTURES FOR RENAL NEUROMODULATION AND ASSOCIATED SYSTEMS AND METHODS,” Pub. No. US-2012-0101413-A1, which is incorporated herein by reference in its entirety.

FIG. 7 is a block diagram illustrating a method 700 of modulating renal nerves using the system 10 described above with reference to FIG. 5. The method 700 can optionally include diagnosing a patient (if not yet determined) and/or selecting a suitable candidate patient for performing renal neuromodulation (block 702). The method 700 can include intravascularly locating the therapeutic assembly or neuromodulation assembly 21 in a delivery state (e.g., low-profile configuration, see FIG. 8A) to a first treatment location. The treatment location may be a target site in or near a first renal blood vessel (e.g., first renal artery) or first renal ostium (block 705). The treatment device 12 and/or portions thereof (e.g., the neuromodulation assembly 21) can be inserted into a guide catheter or sheath to facilitate intravascular delivery of the neuromodulation assembly 21. In certain embodiments, for example, the treatment device 12 can be configured to fit within an 8 Fr guide catheter or smaller (e.g., 7 Fr, 6 Fr, etc.) to access small peripheral vessels. A guide wire (not shown) can be used to manipulate and enhance control of the shaft 16 and the neuromodulation assembly 21 (e.g., in an over-the-wire or a rapid-exchange configuration). In some embodiments, echogenic enhancements, markers or features and/or radiopaque markers and/or markings on the treatment device 12 and/or the guide wire can facilitate placement of the neuromodulation assembly 21 at the first target site (e.g., a first renal artery or first renal ostium of a PKD patient). In some embodiments, a contrast material can be delivered distally beyond the neuromodulation assembly 21, and fluoroscopy and/or other suitable imaging techniques can be used to aid in placement of the neuromodulation assembly 21 at the first target site. In some embodiments, IVUS is used in combination with or alone to ensure placement.

The method 700 can further include connecting the treatment device 12 to the console 26 (block 710), and determining whether the neuromodulation assembly 21 is in the correct position at the target site and/or whether the neuromodulation assembly electrodes or treatment elements are functioning properly (block 715). Determining in block 715 also includes the use of an intravascular imaging modality alone or in combination with another imaging modality to coordinate the position of one or more treatment elements in therapeutic proximity to a target site.

Once the neuromodulation assembly 21 is properly located at the first target site and no malfunctions are detected, the console 26 can be manipulated to initiate application of an energy field to the target site to cause electrically-induced and/or thermally-induced partial or full denervation of the kidney (using the one or more therapy elements borne by the support frame). Accordingly, in one specific example, heating and/or cooling of the neuromodulation assembly 21 causes modulation of renal nerves at the first target site to cause partial or full denervation of the kidney associated with the first target site (block 720).

Optionally, depending upon a patient specific treatment plane, the neuromodulation assembly 21 can then be located at a second treatment location. For example, a target site in or near a second renal blood vessel (e.g., second renal artery) or second renal ostium (block 725), and correct positioning of the assembly 21 can be determined (block 730) as described in 715 with suitable imaging techniques. The method 700 continues by applying targeted therapy effectuate renal neuromodulation at the second target site to cause partial or full denervation of the kidney associated with the second target site (block 735).

After providing the therapeutically-effective neuromodulation energy (e.g., cryogenic cooling, RF energy, microwave, ultrasound energy, etc.), the method 700 may also include determining whether the neuromodulation therapeutically treated the patient for PKD or otherwise sufficiently modulated nerves or other neural structures proximate the first and second target sites (block 740). In one aspect, the method may also include the introduction of one or more pharmacological agents. For example, the process of determining whether the neuromodulation therapeutically treated the nerves can include determining whether nerves were sufficiently denervated or otherwise disrupted to reduce, suppress, inhibit, block or otherwise affect the afferent and/or efferent renal signals. In a further embodiment, patient assessment could be performed at time intervals (e.g., 1 month, 3 months, 6 months, 12 months) following neuromodulation treatment. For example, a PKD patient can be assessed for measurements of perceived pain, blood pressure control, imaging-based measurements of cyst size and number, markers of renal injury (e.g., serum BUN levels, serum creatinine levels, serum cystatin C levels, proteinuria levels, and NGAL and Kim-1 levels), and measures of sympathetic activity (e.g., MSNA, renal and/or total body spillover, plasma norepinephrine levels, and heart rate variability).

In other embodiments, various steps in the method 700 and 1900 can be modified, omitted, and/or additional steps may be added. In further embodiments, the methods can have a delay between applying therapeutically-effective neuromodulation energy to a first target site at or near a first renal artery or first renal ostium and applying therapeutically-effective neuromodulation energy to a second target site at or near a second renal artery or second renal ostium. For example, neuromodulation of the first renal artery can take place at a first treatment session, and neuromodulation of the second renal artery can take place a second treatment session at a later time. As described below, the methods 700 and 1900 may be provided as a revision therapy after a prior treatment has not provided the desired therapeutic results.

As shown in FIG. 8A, the distal end region of the elongated shaft can flex in a substantial fashion due to the shape induced by the stowed spiral support frames. As a result, rotation of the shaft may be used to gain entrance into a respective left/right renal artery by manipulation of the elongated shaft 16. Optionally, the distal end region 20 of the elongated shaft 16 can gain entrance to the renal artery following a path defined by a guide catheter, a guide wire, or a sheath (not shown). In such cases, the maximum outer dimension (e.g., diameter) of any section of the elongated shaft 16, including the therapy element it carries, may be dictated by the inner diameter of the guide catheter through which the elongated shaft 16 is passed. Assuming, for example, that an 8 French guide catheter (which has an inner diameter of approximately 0.091 inch (2.31 mm)) would likely be, from a clinical perspective, the largest guide catheter used to access the renal artery, and allowing for a reasonable clearance tolerance between the elongated shaft 16 and the guide catheter, the maximum outer dimension can realistically be expressed as being less than or equal to approximately 0.085 inch (2.16 mm). However, use of a smaller 5 French guide catheter may require the use of smaller outer diameters along the elongated shaft 16. For example, an elongated shaft 16 that is to be routed within a 5 French guide catheter may have an outer dimension of no greater than 0.053 inch (1.35 mm). In another example, an elongated shaft 16 that is to be routed within a 6 French guide catheter may have an outer dimension of no great than 0.070 inch (1.78 mm). In still further examples, other suitable guide catheters may be used, and outer dimensions and/or arrangements of the treatment device can vary accordingly.

In still other embodiments, there is provided a one time or reusable connection on a proximal end, a distal end, a proximal portion or a distal portion or along one or both support frames a therapy device described herein. The connection port includes at least one communication port to at least one of the lumens of the spiral support frame therapy device. The communication port is suitably configured based on the operation characteristics of the therapy device. If the lumen contains an electrical or electronic component, the connection includes a suitably configured connection. Similarly for mechanically actuated components on the device, a suitable mechanical, magnetic or other suitable form of connection is provided in the connection port. In the same way, any fluid delivery conduit would be suitably fluidly connected via the connection port. In one aspect, the connection port is used as the single connection port to connect the various therapy elements and other components, sensors or devices of the spiral support frame to the proximally positioned support components such as a handle, delivery system for fluids and/or energy delivery system. In one aspect, the connection port remains connected during implantation and initial therapy but is then disconnected and removed. Later, if a patient is to receive an additional therapy, the connection point is advanced to the implant site and coupled to the support frame. Thereafter, appropriate therapy may be delivered using the spiral therapy device via the connection port.

Once entrance to a target site is gained, the treatment element(s) optionally may be aligned with tissue along an interior wall of the lumen using IVUS or other image guidance to align the element(s) to be within therapeutic range of a targeted treatment site. Optionally, the support frame treatment device also may be within the lumen and then employ stabilizing members, prongs, anchors etc. that may be remotely expanded and collapsed or deployed or retrieved via the handle assembly (e.g., FIG. 17B). The spiral support frame device has a low-profile delivery configuration for intravascular delivery to, and retrieval from, within the renal artery (e.g., through a guide catheter), and an expanded deployed configuration (as seen in for example FIGS. 8B, 10A, 10B and 10C) wherein the one or more therapy element contacts the internal luminal surface.

Neuromodulating effects can include thermal ablation, non-ablative thermal alteration, coagulation or damage (e.g., via sustained heating and/or dielectric heating), and electromagnetic neuromodulation. Desired dielectric heating effects may include raising the temperature of target neural fibers above a certain threshold to achieve non-ablative thermal alteration, or above a higher temperature to achieve ablative thermal alteration. For example, treatment devices described herein may be employed where the target temperature can be above body temperature (e.g., approximately 37° C.) but less than about 45° C. for non-ablative thermal alteration, or the target temperature can be about 45° C. or higher for ablative thermal alteration. Desired non-thermal neuromodulation effects may include altering the electrical signals transmitted in a nerve.

It may be desirable to heat the renal nerves disposed within the adventitia while avoiding significant heating of the intima/media during therapy delivery. Renal arterial blood flow may provide passive protective cooling of the intima/media. Thus, by maintaining therapy elements against the arterial wall the spiral frame devices herein do not obstruct the vessel lumen. Devices described herein ensure continued blood flow cooling of the renal artery intima/media during treatment of target renal nerves or sites. In some multi-conduit devices, one or more than one conduits or apertures in a conduit may increase the velocity of blood flow at or near the vessel wall to enhance or accelerate the transfer of heat from the wall to the blood using a perfusion technique. FIGS. 16A, 16B, 16C, 16D, 16E, 16F and 17B illustrate a wide variety of multi-conduit configurations for the spiral frame therapy devices described herein.

In still other various embodiments, embodiments of the therapeutic devices described herein may be adapted or configured according to one or more of the devices, components or configurations described in U.S. patent application Ser. No. 13/926,935, filed Jun. 25, 2013, titled “RENAL DENERVATION AND STIMULATION EMPLOYING WIRELESS VASCULAR ENERGY TRANSFER ARRANGEMENT,” Pub. No. US-2013-0274735-A1, which is incorporated herein by reference in its entirety. While the conduits of the multiple conduit system have been described as carrying or providing advantageous positioning and use for fixation elements, ablation elements and delivery of fluids, the conduits described herein and the multi-function aspects of the device embodiments are not so limited. In addition or alternatively, one or more conduits may be adapted and configured to house portions of an electronic system used to control operation or one or more components borne by or connected to a therapy device. In one specific example a conduit or portion thereof may be used to house an antenna or a coil or a portion of a transcutaneous receiver or communications system or transcutaneous power coupling or transfer system or other components. As is clear from the various illustrative embodiments of the various multiple conduit therapy devices, the number, size, placement, relative orientation and position of the one or more conduits may be modified to accomplish the desired functionality. In one specific aspect, one or more conduits or a portion there of us used to house or provide access to: a flexible electrical connector, an impedance sensor, a pressure sensor, a temperature sensor, an antenna, a wire coil, a sensing circuit, a circuit for sensing or recording a biometric signal, a circuit for detecting a change in a biometric signal and providing an output based on the change, a control circuit, a rechargeable battery, a timing circuit, a detector, a component adapted and configured to be controlled by an external control device, a component adapted and configured to be controlled by an internal or implanted control device, a component adapted and configured to be controlled via a wired connection to a control device, a component adapted and configured to be controlled via wireless connection to a control device, as well as other components or devices suited to providing one or more of the different therapy modes or positions described herein.

In FIG. 17B a spiral frame device comprises a plurality of (e.g., fingers or prongs), which are connected to the handle for extension or retraction.

In one embodiment of FIG. 8A, in the low-profile delivery configuration, the outer sheath serves as a delivery sheath thereby constraining normally deployed anchors as described in U.S. patent application Ser. No. 13/553,335, filed Jul. 19, 2012, titled “ENDOLUMINAL FILTER WITH FIXATION,” Publ. No. US-2013-0012981-A1 and International Pat. App. No. PCT/US2008/088606, filed Dec. 31, 2008, titled “ENDOLUMINAL FILTER WITH FIXATION,” Pub. No. WO 2009/088905, each of which is incorporated herein by reference in its entirety.

As seen in FIG. 17B, upon proximal retraction of the delivery sheath (e.g., via actuation of the handle assembly 200), the anchors self-expand into contact with or completely or partially through the vessel wall, thereby anchoring the therapy device in the vessel.

In an alternative embodiment of an anchored support frame delivery device, the anchor elements may self-expand into contact with the vessel wall and/or may be actively expanded via actuation at the handle. Embodiments comprising expandable anchor elements may have a multiple number (e.g., two, three, four, five, etc.) along all or a portion of a frame. Anchors may have variations of geometric shapes (e.g., straight, curved, helical, coiled, etc.). Anchors may also be configured for reversible actuation. Optionally or in addition, one or more elements delivered via a multi conduit support frame embodiment may be configured for therapy according to U.S. patent application Ser. No. 13/294,439, filed Nov. 11, 2011, titled “EXPANDABLE CATHETER SYSTEM FOR VESSEL WALL INJECTION AND MUSCLE AND NERVE FIBER ABLATION,” Pub. No. 2013/0053792, which is herein incorporated by reference in its entirety.

Various design features of the spiral support frame embodiments described herein may be adapted and configured to address the cellular misalignment of the renal nerves and the smooth muscle cells may be exploited to selectively affect renal nerve cells with reduced effect on smooth muscle cells. More specifically, because larger cells require a lower electric field strength to exceed the cell membrane irreversibility threshold voltage or energy for irreversible electroporation, embodiments of therapy element positioning along the spiral support frame or by positioning of the support frame of the present invention may be configured to align at least a portion of an electric field generated by the therapy elements with or near the longer dimensions of the cells to be affected. In specific embodiments, the device has electrodes configured to create an electrical field aligned with or near the lengthwise dimension of the renal artery RA to affect renal nerves RN. By aligning an electric field so that the field preferentially aligns with the lengthwise aspect of the cell rather than the diametric or radial aspect of the cell, lower field strengths may be used to affect target neural cells, e.g., to necrose or fuse the target cells, to induce apoptosis, to alter gene expression, to attenuate or block action potentials, to change cytokine up-regulation and/or to induce other suitable processes. This is expected to reduce total energy delivered to the system and to mitigate effects on non-target cells in the electric field. These and other optioned or alternative aspects are described in U.S. Pat. No. 8,150,520 which is herein incorporated by reference in its entirety.

Similarly, the lengthwise or longer dimensions of tissues overlying or underlying the target nerve are orthogonal or otherwise off-axis (e.g., transverse) with respect to the longer dimensions of the nerve cells. Thus, in addition to aligning energy delivery with the lengthwise or longer dimensions of the target cells, the delivered energy may propagate along the lateral or shorter dimensions of the non-target cells (i.e., such that the energy propagates at least partially out of alignment with non-target smooth muscle cells SMC).

In one aspect, a therapy element may be one or more electrodes configured, for example, as individual electrodes (i.e., independent contacts), a segmented electrode with commonly connected contacts, or a single continuous electrode. Furthermore, the electrodes may be configured to provide a bipolar signal, or the electrodes may be used together or individually in conjunction with a separate patient ground pad for monopolar use. As an alternative or in addition to placement of the electrodes along the support elements of the spiral support, the electrodes may be attached such that each has variable contact the wall of the renal artery RA (see FIGS. 15 and 15C). In other variations, the electrodes or other therapy elements may, for example, be affixed to the inside surface, outside surface or at least partially embedded within the wall of the support frame (see e.g., FIGS. 6B, 6C, 6D, 10A, 10B, 10C, 11A, 11C, 12A-12H, or 13A and 13B).

FIGS. 6B, 6C and 6D illustrate a spiral support frame embodiment having a plurality of evenly spaced therapy elements 135 along the length of one or both of the spiral support members. FIG. 10B illustrates a single therapy element 135 positioned along the length of one of the support frames of a spiral support frame. In this illustrative embodiment, the therapy elements are on support member 105. This embodiment could also be configured on support member 110. FIG. 10B illustrates a series of individually controllable single therapy elements 135 positioned along the length of one of the support frames of a spiral support frame. In this illustrative embodiment, the therapy elements are on support member 105. This embodiment could also be configured on support member 110. FIG. 10C illustrates a series of individually controllable single therapy elements 135 positioned along the length of one of the support frames in only one loop of a spiral support frame. In this illustrative embodiment, the therapy elements are on support member 110. This embodiment could also be configured on support member 105. While illustrated on one side of one loop adjacent proximal end 104, other configurations are possible adjacent to distal end 102.

FIG. 11A and 11B illustrate a series of individually controllable single therapy elements 135 positioned in a staggered or offset position along the length of each one of the support frames 105, 110 of a spiral support frame. FIG. 11A illustrates the deployed configuration where the spiral support frame is expanded so as to engage the individual therapy elements 135 with an adjacent luminal wall. FIG. 11B illustrates the FIG. 11A embodiment in a stowed configuration. The view of FIG. 11B illustrates how the offset spacing of the therapy elements permits the therapy elements borne by support member 105 to lie between without contacting the therapy elements borne by support member 110.

FIGS. 11C and 12A -12E illustrate a variety of different configurations and types of therapy elements. It is to be appreciated that one or more of the types of different therapy element and support frame configurations may be used in a particular embodiment depending upon the type of therapy being provided.

FIG. 11C illustrate a series of individually controllable single therapy elements 135 positioned in a staggered or offset position along the length of each one of the support frames 105, 110 of a spiral support frame. In contrast to the embodiment of FIGS. 11A, 11B, the therapy elements in this embodiment are only partially encircling the support member. In the embodiments of FIGS. 11A, 11B the therapy elements completely encircle the support frame.

FIGS. 12A-12E are cross section views of a support frame 105 and a therapy element 135 according to various different embodiments. Each may also be on the support frame 110 alone or on both of the support frames 105, 110. Each of the therapy elements may be configured in one or more than one segments on one or both of the support frames as in FIGS. 10A, 10B, 10C and 11A. In each of the various views the connection from the therapy element may be provided via the support frame or via a multiple lumen sheath embodiment (see FIGS. 14-16F) but are omitted here for clarity. Still further alternatives having therapy elements that are less than a full circumference of the support frame (i.e., where the therapy element has an orientation with respect to the support frame) the therapy element may be positioned relative to the support frame wherein—in the deployed configuration—a therapy element is against the lumen wall, directed towards the lumen interior (i.e., to measure or monitor a fluid flowing through the lumen, for example) or in a position between the lumen wall and the lumen flow path. It is to be appreciated that the illustrative embodiments may be modified to represent each of these various different orientations for the placement of a therapy element so as to be oriented on the support frame relative to the lumen interior wall and lumen flow.

FIG. 12A is a cross section view of a generally cylindrical therapy element 135 adjacent to a generally cylindrical support frame 105.

FIG. 12B is a cross section view of a generally half elliptical therapy element 135 embedded into a generally rectangular support frame 105.

FIG. 12C is a cross section view of a pair of elliptical therapy elements 135 embedded into a generally rounded rectangular support frame 105.

FIG. 12D is a cross section view of a generally rectangular therapy element 135 embedded into a generally semicircular support frame 105.

FIG. 12E is a cross section view of a ring or annular therapy element 135 on a generally cylindrical support frame 105.

It is to be appreciated that the above described therapy elements and relative configurations may also be arrayed into different configurations along the length of one or both of the support frames. For example, FIG. 12F illustrates a perspective view of a portion of a support frame having a linear array of regularly sized and spaced therapy elements along a portion of a support frame. In still other aspects, as shown in FIG. 12G, there is a perspective view of a portion of a support frame having an irregular dispersion pattern of similarly sized therapy elements along a portion of a support frame. In still other aspects, a portion of one or more therapy elements may be configured to penetrate partially or completely through an adjacent lumen interior wall. FIG. 12H illustrates a perspective view of a portion of a support frame having a pair of spaced apart penetrating therapy elements. The length and shape of a penetrating therapy element may be adapted according to the various anatomical requirements of a particular intraluminal therapy site.

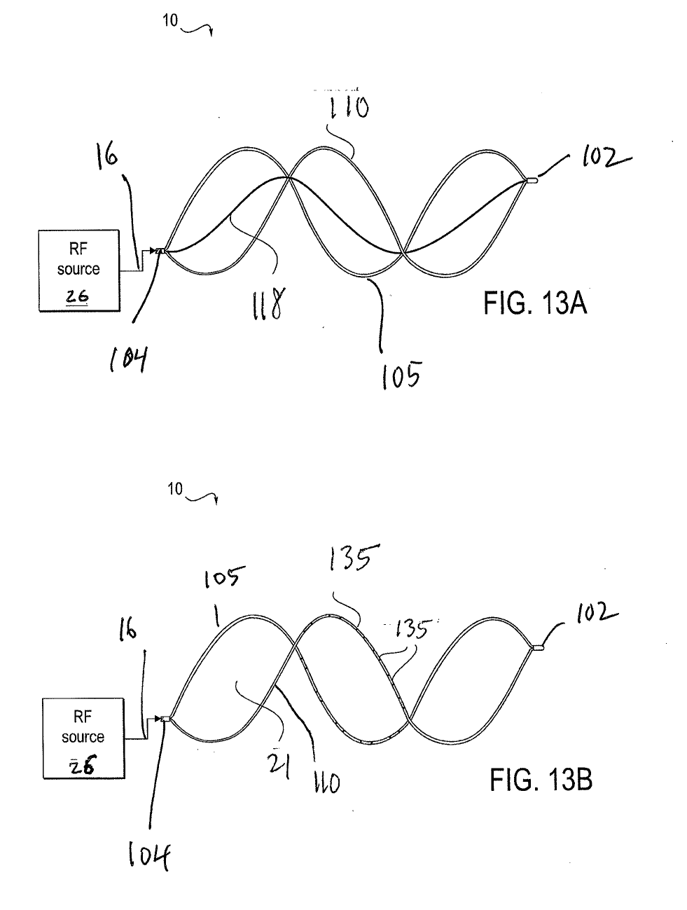

The spiral support frame therapy device may have more than two loops. FIG. 13A illustrates a multiple loop support frame having a therapy element extending from the proximal end to the distal end of the support frame. In this embodiment, the support frames 105, 110 are formed into three loops. A separate element 118 is provided along and between the loops. In this embodiment, one or more therapy elements as described herein are borne by the element 118. FIG. 13B illustrates another multiple loop support frame having a plurality of therapy elements placed only on one of the support frame loops. In this embodiment, the support frames 105, 110 form three loops. The middle loop between the two crossovers contains the one or more therapy elements 135. While illustrating therapy elements 135 along both of the support frames 105, 110 other configurations are possible as described elsewhere herein.

The one or more therapy elements optionally may be used to monitor the effects of a therapy. As it may be desirable to reduce or minimize physical contact between the therapy elements and the vessel wall during delivery of therapy, e.g., to reduce the potential for injuring the wall, the therapy element wall contact force may be adjusted by the characteristics and properties of the spiral support frame. The orientation of the therapy element relative to the deployed support frame/lumen wall position may also be used.

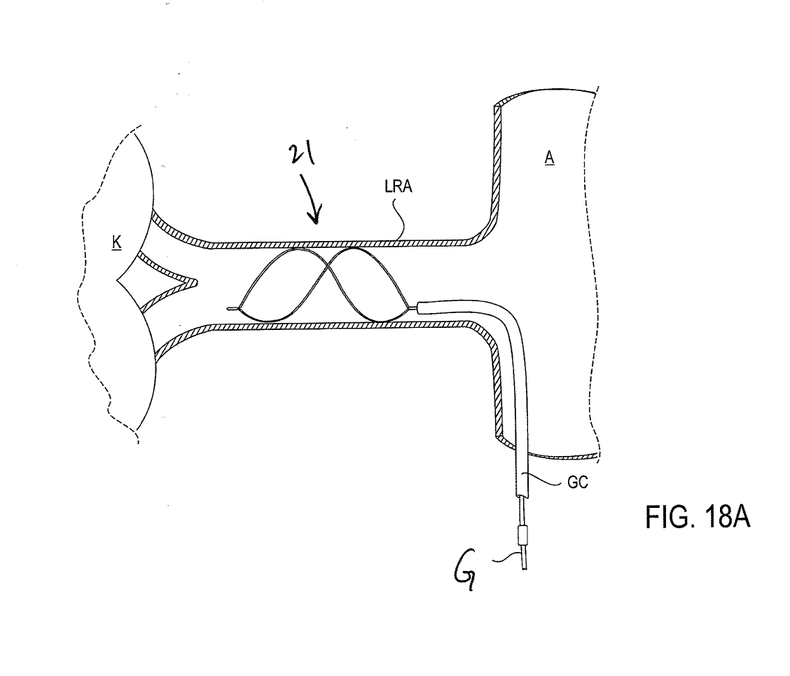

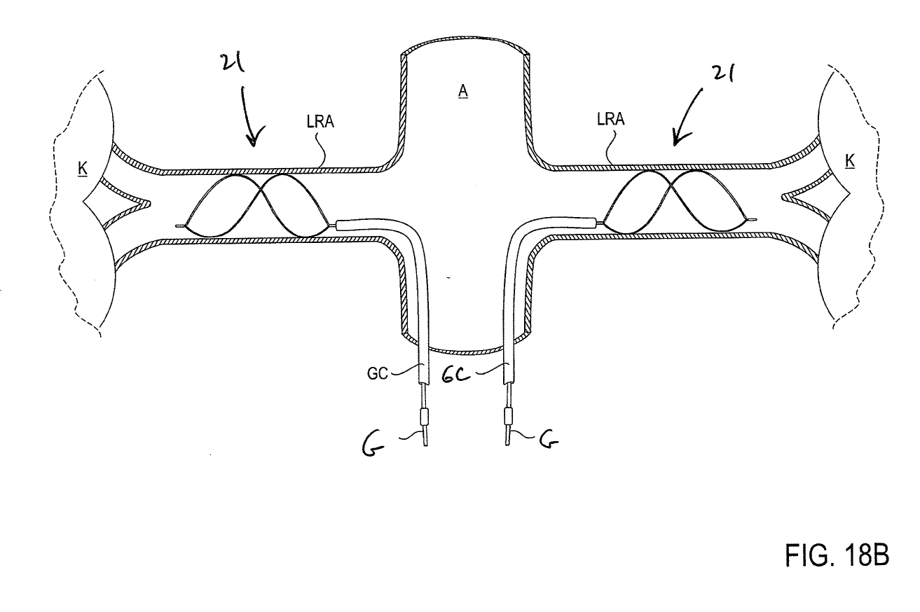

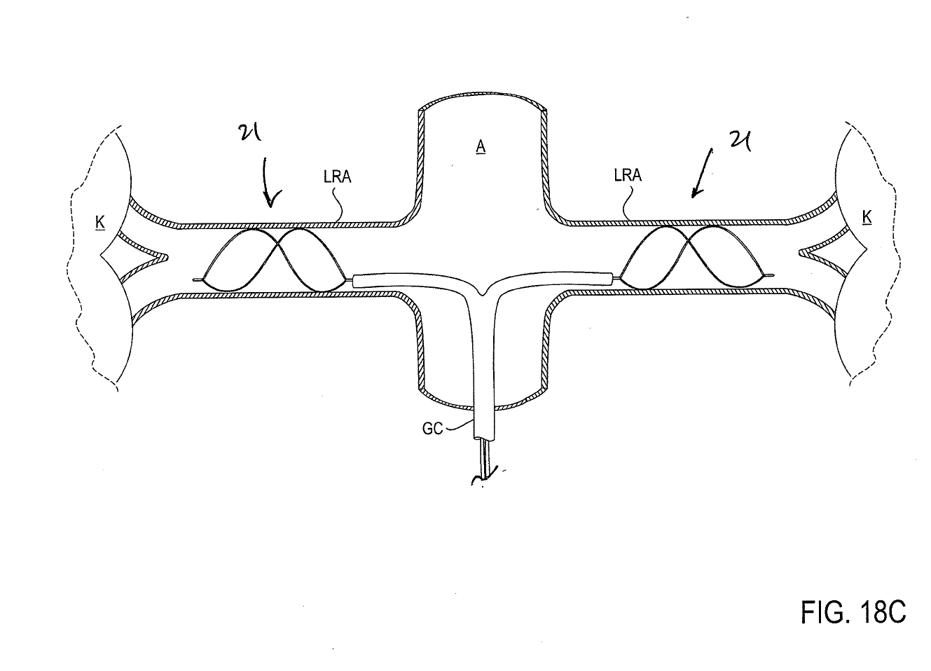

It may be desirable to achieve unilateral or bilateral single mode or multiple mode therapy configured for renal neuromodulation. Bilateral neuromodulation may enhance the therapeutic effect in some patients as compared to renal neuromodulation performed unilaterally, i.e., as compared to renal neuromodulation performed on neural tissue innervating a single kidney. For example, bilateral renal neuromodulation may further reduce clinical symptoms of CHF, hypertension, acute myocardial infarction, contrast nephropathy, renal disease and/or other cardio-renal diseases. Still further, delivery of stimulation energy before, during or after perfusion may also be beneficial. FIGS. 18A, 18B and 18C illustrate multi-mode spiral support frame therapy devices for use according to multi-mode, multi-location method 1900.

In one aspect, a guide catheter GC and a guidewire G may be advanced into position within, or in proximity to, either the patient's left renal artery LRA or right renal artery RRA. The guidewire may be positioned in the right renal artery RRA, but it should be understood that the order of bilateral renal neuromodulation may be reversed. Additionally or alternatively, bilateral renal neuromodulation may be performed concurrently on both right and left neural fibers that contribute to renal function, rather than sequentially.

With the guidewire and the guide catheter positioned in the right renal artery, the spiral frame apparatus may be advanced over the guidewire and through the guide catheter into position within the artery. As seen in FIG. 18A, once the multi-mode spiral frame delivery device is properly positioned for therapy, the support frame is expanded to bring therapy elements into contact with the vessel wall, and the guidewire G may be retracted from the treatment zone, e.g., may be removed from the patient or may be positioned more proximally within the patient's aorta. Thereafter, therapy proceeds as in method 1900.

After completion of the therapy, the multi-mode spiral support frame may be collapsed back to the reduced delivery profile, and retracted from the right renal artery RRA, for example, to a position in the guide catheter GC within the patient's abdominal aorta for removal or for positioning in another RRA treatment site or the left renal artery. Likewise, the guide catheter GC may be retracted to a position within the patient's aorta. The retracted frame may be repositioned, e.g., rotated, such that it is generally aligned with the left renal artery LRA. The support frame with or without the guidewire G then may be re-advanced to a position within the left renal artery LRA. As will be apparent, the order of advancement of the guidewire and the support frame optionally may be reversed when accessing either renal artery.

As discussed previously, bilateral renal neuromodulation optionally may be performed concurrently on fibers that contribute to both right and left renal function. FIGS. 18B and 18C illustrate embodiments of multi-mode or single mode spiral supports for performing concurrent bilateral renal neuromodulation. In the embodiment of FIG. 18B, apparatus comprises dual therapy support frames, as well as dual guidewires G and guide catheters GC if needed. One support frame therapy device is positioned within the right renal artery RRA, and the other is positioned within the left renal artery LRA. With support frame devices positioned in both the right and left renal arteries, single mode or multi-mode therapy may be delivered concurrently by the support frame to achieve concurrent bilateral renal neuromodulation, illustratively via an intravascular approach.

In one example, separate arteriotomy sites may be made in the patient's right and left femoral arteries for percutaneous delivery of the two support frames. Alternatively, both support frames may be delivered through a single femoral access site, either through dual guide catheters or through a single guide catheter. FIG. 18C illustrates an example of apparatus for concurrent bilateral renal neuromodulation utilizing a single arteriotomy access site. In the example of FIG. 18C, both support frames are delivered through a bifurcated guide catheter having a bifurcated distal region for concurrently delivering the support frames to the right and left renal arteries. Concurrent (or sequential) bilateral, single or multiple mode therapy then may proceed.

Turning now to FIG. 19 and the method 1900, the method 1900 describes a multiple therapy mode delivery method similar to the therapy method described in FIG. 7 and the method 700. FIG. 19 is a block diagram illustrating a method 1900 of providing a multiple mode therapeutic assembly into one or more treatment locations for delivery one or more modes of therapy. The method 1900 can optionally include diagnosing a patient (if not yet determined) and/or selecting a suitable candidate patient for performing a multiple therapy location and/or multiple mode therapy (block 1902). One therapy mode may include renal neuromodulation. The method 1900 can include locating a therapeutic assembly of a multiple mode treatment device or neuromodulation assembly in a delivery state (e.g., low-profile configuration, see FIG. 8A) to a first treatment location. (block 1905). The treatment location may be a target site in or near a first renal blood vessel (e.g., first renal artery) or first renal ostium (block 705). The treatment location may also be another point accessed by the arterial or venous vasculature where one or more modes of therapy is to be delivered using one or more delivery modes. The therapeutic assembly and/or portions thereof (e.g., the neuromodulation assembly) can be inserted into a guide catheter or sheath to facilitate intravascular delivery to the first and subsequent treatment locations. In certain embodiments, for example, the treatment device can be configured to fit within an 8 Fr guide catheter or smaller (e.g., 7 Fr, 6 Fr, etc.) to access small peripheral vessels. A guide wire (not shown) can be used to manipulate and enhance control of the shaft and a neuromodulation assembly 21 (e.g., in an over-the-wire or a rapid-exchange configuration). In some embodiments, echogenic enhancements, markers or features and/or radiopaque markers and/or markings on the treatment device and/or the guide wire can facilitate placement of the neuromodulation assembly at the first target site (e.g., a first renal artery or first renal ostium of a PKD patient). In some embodiments, a contrast material can be delivered distally beyond the neuromodulation assembly, and fluoroscopy and/or other suitable imaging techniques can be used to aid in placement of the neuromodulation assembly 21 at the first target site. In some embodiments, IVUS is used in combination with or alone to ensure placement. In still other embodiments, an imaging contrast media is delivered via one or more of the conduits (e.g., FIG. 15A-16C, or 17B).

The method 1900 can further include connecting the multiple mode treatment device to the console 26 or additional therapy delivery device depending upon mode to be delivered (block 1910). Thereafter, there is a step of determining whether the neuromodulation assembly 21 is in the correct position at the target site and/or whether the neuromodulation assembly electrodes or treatment elements are functioning properly (block 1915). Determining in block 1915 also includes the use of an intravascular imaging modality alone or in combination with another imaging modality to coordinate the position of one or more treatment elements in therapeutic proximity to a target site. This step is repeated or adjusted depending upon the specific location of the therapy mode to be used in the treatment step. For example, if a drug is to be delivered via a series of delivery ports in a particular position on the device. Then this step would be used to ensure appropriate placement of the delivery ports.

Once the neuromodulation assembly 21 is properly located at the first target site and no malfunctions are detected, the console 26 can be manipulated to initiate application of an energy field to the target site to cause electrically-induced and/or thermally-induced partial or full denervation of the kidney (using the one or more therapy elements borne by the support frame). Accordingly, in one specific example, heating and/or cooling of the neuromodulation assembly 21 causes modulation of renal nerves at the first target site to cause partial or full denervation of the kidney associated with the first target site (block 1920). The first treatment may be in a different location or use a different therapy mode, in other embodiments.

Optionally, depending upon a patient specific treatment plan, the neuromodulation assembly 21 can then be used to for delivery of another or additional treatments at the same or with different therapy modalities at the first treatment site (block 1925).

Next, according to method 1900, the therapeutic assembly is positioned in a second treatment location (block 1930). For example, a second treatment location may be a target site in or near a second renal blood vessel (e.g., second renal artery) or second renal ostium, and correct positioning of the assembly 21 can be determined (block 1935) with suitable imaging or other positioning techniques depending upon the type of therapeutic device in use. The method 1900 continues by applying a first therapy mode at the second treatment location. The second therapy mode may be used, for example, as part of a targeted therapy to effectuate renal neuromodulation at the second target site. In still other embodiments, the therapy may be used to cause partial or full denervation of the kidney associated with the second target site (block 1940). Thereafter, a second delivery of the first mode of therapy or a delivery of a different therapy mode is provided to the second therapy site (1945).

After providing the first and second modes and the first and second therapy sites, the method 1900 may then proceed to determine whether there was sufficient treatment (block 1950). If additional treatments are needed, then the methods of FIG. 7 or FIG. 19 may be repeated or modified based upon the newly evaluated patient condition. In one aspect, the first and second treatment modalities are combined to provide therapeutically-effective neuromodulation energy (e.g., cryogenic cooling, RF energy, ultrasound energy, etc.). The method 1900 may also include determining whether the neuromodulation therapeutically treated the patient for PKD or otherwise sufficiently modulated nerves or other neural structures proximate the first and second target sites (blocks 1920, 1925, 1940, 1945). For example, the process of determining whether the neuromodulation therapeutically treated the nerves can include determining whether nerves were sufficiently denervated or otherwise disrupted to reduce, suppress, inhibit, block or otherwise affect the afferent and/or efferent renal signals. In a further embodiment, patient assessment could be performed at first or second locations using one or both or other combinations of treatment modes at time intervals (e.g., 1 month, 3 months, 6 months, 12 months) following neuromodulation treatment. For example, a PKD patient can be assessed for measurements of perceived pain, blood pressure control, imaging-based measurements of cyst size and number, markers of renal injury (e.g., serum BUN levels, serum creatinine levels, serum cystatin C levels, proteinuria levels, and NGAL and Kim-1 levels), and measures of sympathetic activity (e.g., MSNA, renal and/or total body spillover, plasma norepinephrine levels, and heart rate variability).

In other embodiments, various steps in the method 700 and 1900 can be modified, omitted, and/or additional steps may be added. In further embodiments, the methods can have a delay between applying therapeutically-effective neuromodulation energy to a first target site at or near a first renal artery or first renal ostium and applying therapeutically-effective neuromodulation energy to a second target site at or near a second renal artery or second renal ostium. For example, neuromodulation of the first renal artery can take place at a first treatment session, and neuromodulation of the second renal artery can take place a second treatment session at a later time. As described below, the methods 700 and 1900 may be provided as a revision therapy after a prior treatment has not provided the desired therapeutic results.

In one example of methods and apparatus for achieving neuromodulation, localized drug delivery is provided by a drug reservoir, optionally part of an implantable drug pump, has been implanted within the patient. A multi-conduit support frame embodiment is configured then as a type of drug delivery catheter and connected to the drug reservoir and extend to the vicinity of a treatment site, such as right renal artery RRA and the left renal artery LRA, respectively, for delivery of one or more neuromodulatory agents or drugs capable of modulating neural fibers that contribute renal function. Delivering the agent(s) as described herein may achieve bilateral renal neuromodulation. Such drug delivery through multi conduit devices may be conducted concurrently or sequentially, as well as continuously or intermittently, as desired, with other modalities described herein in order to provide concurrent or sequential, continuous or intermittent, renal neuromodulation, respectively.

In an alternative embodiment of the apparatus of FIGS. 16A, 18A, 18B and 18 catheters 402a and 402b may only temporarily be positioned at a desired location, e.g., for acute delivery of the neuromodulatory agent(s) from an external drug reservoir, such as a syringe. Such temporary positioning may comprise, for example, intravascular, extravascular and/or intra-to-extravascular placement of the catheters. In another alternative embodiment, a therapy system may be provided with an implantable neurostimulator or a pacemaker-type device. Electrical leads coupled to the neurostimulator for delivery of an electric field, such as a pulsed electric field or a stimulation electric field, to the target neural fibers via one or more support frame borne treatment elements. In yet another alternative embodiment, electrical techniques may be combined with delivery of neuromodulatory agent(s) to achieve desired bilateral renal neuromodulation.

Although preferred illustrative variations of the present invention are described above, it will be apparent to those skilled in the art that various changes and modifications may be made thereto without departing from the invention. For example, although the variations primarily have been described for use in combination with pulsed electric fields, it should be understood that any other electric field may be delivered as desired, including stimulation or nerve block electric fields, and any other alternative neuromodulatory techniques, such as localized delivery of a neuromodulatory agent or drug, may be utilized. Additionally or optionally, the system devices and methods described herein may be used with pulsed electronic field therapy described below.

Congestive Heart Failure (“CHF”) is a condition that occurs when the heart becomes damaged and reduces blood flow to the organs of the body. If blood flow decreases sufficiently, kidney function becomes altered, which results in fluid retention, abnormal hormone secretions and increased constriction of blood vessels. These results increase the workload of the heart and further decrease the capacity of the heart to pump blood through the kidneys and circulatory system. Numerous systems have been described for such therapies. In another embodiment, the methods, devices and systems described herein are utilized as a type of revision or follow on therapy. In this aspect, a patient is treated using a first type of renal denervation therapy and subsequently evaluated. The result of the evaluation identifies one or more additional target sites for additional treatment. The anatomical locations of such additional sites are then used as the target sites in the methods herein (e.g., methods 700 and 1900) where the IVUS capabilities of the system ensure localized placement to that the identified failed, revision or augmented therapy sites.

In addition or alternatively, described methods and apparatus for treating renal disorders by applying a pulsed electric field to neural fibers that contribute to renal function. See, for example, U.S. patent application Ser. No. 11/129,765, filed May 13, 2005 (now U.S. Pat. No. 7,653,438), and U.S. patent application Ser. No. 11/189,563, filed Jul. 25, 2005 (now U.S. Pat. No. 8,145,316), both of which are incorporated herein by reference in their entireties. A pulsed electric field (“PEF”) may initiate renal neuromodulation, e.g., denervation, for example, via irreversible electroporation or via electrofusion may also be considered within the treatment modalities of the methods and systems described herein. The PEF may be delivered from apparatus positioned intravascularly, extravascularly, intra-to-extravascularly or a combination thereof in accordance with intraluminal and/or transluminal therapy delivery. Additional methods and apparatus for achieving renal neuromodulation, e.g., via localized drug delivery (such as by a drug pump or infusion catheter via a multi conduit device herein) or via use of a stimulation electric field, etc., are described herein and, for example, U.S. patent application Ser. No. 10/408,665, filed Apr. 8, 2003 (now U.S. Pat. No. 7,162,303), and U.S. Pat. No. 6,978,174, both of which are incorporated herein by reference in their entireties.

As used herein, electrofusion comprises fusion of neighboring cells induced by exposure to an electric field. Contact between target neighboring cells for the purposes of electrofusion may be achieved in a variety of ways, including, for example, via dielectrophoresis. In tissue, the target cells may already be in contact, thus facilitating electrofusion.

As used herein, electroporation and electropermeabilization are methods of manipulating the cell membrane or intracellular apparatus. For example, the porosity of a cell membrane may be increased by inducing a sufficient voltage across the cell membrane through, e.g., short, high-voltage pulses. The extent of porosity in the cell membrane (e.g., size and number of pores) and the duration of effect (e.g., temporary or permanent) are a function of multiple variables, such as field strength, pulse width, duty cycle, electric field orientation, cell type or size and/or other parameters.

Cell membrane pores will generally close spontaneously upon termination of relatively lower strength electric fields or relatively shorter pulse widths (herein defined as “reversible electroporation”). However, each cell or cell type has a critical threshold above which pores do not close such that pore formation is no longer reversible; this result is defined as “irreversible electroporation,” “irreversible breakdown” or “irreversible damage.” At this point, the cell membrane ruptures and/or irreversible chemical imbalances caused by the high porosity occur. Such high porosity can be the result of a single large hole and/or a plurality of smaller holes.

A potential challenge of using intravascular PEF systems for treating renal disorders is to selectively electroporate target cells without affecting other cells. For example, it may be desirable to irreversibly electroporate renal nerve cells that travel along or in proximity to renal vasculature, but it may not be desirable to damage the smooth muscle cells of which the vasculature is composed. As a result, an overly aggressive course of PEF therapy may persistently injure the renal vasculature, but an overly conservative course of PEF therapy may not achieve the desired renal neuromodulation.

A number of methods and apparatus for monitoring tissue impedance or conductivity to determine the effects of pulsed electric field therapy, e.g., to determine an extent of electroporation and/or its degree of irreversibility exist, see for example, U.S. patent application Ser. No. 11/233,814, filed Sep. 23, 2005, titled “METHODS AND APPARATUS FOR INDUCING, MONITORING AND CONTROLLING RENAL NEUROMODULATION,” Publication No. US-2007-0083239 A1, which is incorporated herein by reference in its entirety. Pulsed electric field electroporation of tissue causes a decrease in tissue impedance and an increase in tissue conductivity. If induced electroporation is reversible, tissue impedance and conductivity should approximate baseline levels upon cessation of the pulsed electric field. However, if electroporation is irreversible, impedance and conductivity changes should persist after terminating the pulsed electric field. Thus, monitoring the impedance or conductivity of target and/or non-target tissue may be utilized to determine the onset of electroporation and to determine the type or extent of electroporation. Furthermore, monitoring data may be used in one or more manual or automatic feedback loops to control the electroporation. It is to be appreciated that the various support frame therapy devices and systems may be modified to provide one or more of the herein described PEF therapy delivery capabilities.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1.-82. (canceled)

83. An implantable treatment apparatus, comprising:
an elongated shaft having a proximal portion, a distal portion having a spiral support frame;
at least one treatment element borne by the support frame, wherein the support frame is configured for delivery to and implantation within a human patient adjacent to a treatment location; and
wherein the treatment element is configured for positioning against a portion of the anatomy of the patient in a position suited to the therapeutic range or therapy modality of the at least one treatment element borne by the support frame.

84. The implantable treatment apparatus of claim 83, wherein the treatment location is accessible and the support frame is configured for delivery via an artery or a vein.

85. The implantable treatment apparatus of claim 83, wherein the treatment location is accessible and the support frame is configured for delivery via a minimally invasive, laparoscopic or arthroscopic or single port access delivery system.

86. The implantable treatment apparatus of claim 83, wherein the treatment location is accessible and the support frame is configured for delivery via a lumen or natural orifice of the patient.

87. An intraluminal treatment apparatus, comprising:
an elongated shaft having a proximal portion in communication with an energy source and a distal portion coupled to a spiral support frame;
at least one treatment element borne by the spiral support frame and connected via the elongated shaft to the energy source, wherein the support frame is configured for intravascular delivery to a transluminal therapy site in a patient; and
wherein the treatment element is configured for positioning against an interior wall of the lumen to modulate, stimulate or ablate a neural tissue at the transluminal treatment site in the patient.

88. The intraluminal treatment apparatus of claim 87, wherein the at least one treatment device is positioned on one side of a crossover of the spiral support frame.

89. The intraluminal treatment apparatus of claim 87, wherein the at least one treatment device further comprises at least one treatment device on a first spiral support member of the spiral support frame and at least one treatment device on a second spiral support member of the spiral support frame.

90. The intraluminal treatment apparatus of claim 87, wherein the at least one treatment device further comprises at least one treatment device on a first spiral support member comprising a plurality of individually controlled treatment devices on a first spiral support member of the spiral support frame and at least one treatment device on the second spiral support member comprising a plurality of individually controlled treatment devices on a second spiral support member of the spiral support frame.

91. A method for spiral support frame based stimulation, neuromodulation, or ablation, comprising:
positioning a spiral support frame having a therapeutic element within the vasculature of a human patient; and
reducing neural traffic to and/or from or along one or more nerves adjacent the vasculature via the therapeutic element, wherein reducing the neural traffic to and/or from or along the nerves therapeutically treats a diagnosed condition or disease associated with the one or more nerves of the patient.

92. The method of claim 91, wherein positioning a spiral support frame therapeutic device within a patient comprises positioning a catheter using fluoroscopic guidance.

93. The method of claim 91, wherein positioning a spiral support frame therapeutic device within a patient comprises positioning a catheter using intra vascular ultrasound.

94. The method of claim 91, wherein reducing neural traffic comprises transferring energy via the spiral support frame.

95. The method of claim 94, wherein transferring energy via the spiral support frame comprises ablating one or more nerves or a portion thereof in proximity to the spiral support frame.

96. The method of claim 94, wherein transferring energy via the spiral support frame comprises delivering radiofrequency energy via an element borne by the support frame or extending from a conduit of the support frame.

97. The method of claim 91, wherein reducing neural traffic to and/or along comprises blocking neural traffic to and/or from an organ of the patient.

98. The method of claim 91, wherein reducing neural traffic comprises denervating an organ of the patient.

99. The method of claim 91, wherein reducing neural traffic comprises modulating sympathetic nerves that innervate an organ of the patient.

100. The method of claim 91, wherein the step of positioning the spiral support frame further comprises positioning the spiral support frame having the therapeutic element within a renal artery of the human patient; and
wherein the step of reducing neural traffic further comprises reducing neural traffic to and/or from a kidney of the patient via the therapeutic element, wherein reducing the neural traffic to and/or from the kidney therapeutically treats a diagnosed condition or disease associated with cardio-renal function of the patient.

101. The method of claim 91, further comprising:
positioning the spiral support frame having the therapeutic element within a renal artery of the human patient; and
ablating nerves that innervate a kidney of the patient via the therapeutic element, wherein ablating the nerves results in a therapeutically beneficial reduction in blood pressure in the patient.

102. The method of claim 91, further comprising ablating nerves that innervate a portion of the vasculature of the patient via the therapeutic element, wherein ablating the nerves results in a therapeutically beneficial reduction in a targeted central sympathetic overactivity in the patient.
